(19) United States
(12) Patent Application Publication
(10) Pub. No.: US 2025/0268477 A1
(43) Pub. Date: Aug. 28, 2025

(54) DETERMINING A PHYSIOLOGICAL PARAMETER USING OPTICAL SIGNALS

(71) Applicant: QUALCOMM Incorporated, San Diego, CA (US)

(72) Inventors: Changting XU, San Jose, CA (US); Hrishikesh Vijaykumar PANCHAWAGH, Cupertino, CA (US); Bernard HERRERA SOUKUP, Sunnyvale, CA (US)

(21) Appl. No.: 18/584,978

(22) Filed: Feb. 22, 2024

(51) Int. Cl. A61B 5/02; A61B 5/00; A61B 5/021; A61B 8/04

(52) U.S. Cl. CPC A61B 5/02007; A61B 5/0066; A61B 5/02125; A61B 5/6802; A61B 8/04

(57) ABSTRACT

Systems for determining a physiological parameter using optical signals from different light sources are disclosed. Such systems may be embodied in a wearable user device, which may include a first light source system configured to transmit a first light source system configured to transmit one or more first optical signals toward the target object, the one or more second optical signals configured to generate one or more acoustic signals from the a target object; a second light source system configured to transmit a second optical signal toward a skin of the user, the skin being proximate to the target object of the user; and a receiver configured to detect a reflection of the second optical signal from the skin of the user during the generation of the one or more acoustic signals, the reflection of the second optical signal indicative of a physiological parameter of the user.

### TECHNICAL FIELD

This disclosure relates generally to devices and systems using biometric sensors, including contactless biometric sensors.

### DESCRIPTION OF RELATED TECHNOLOGY

A variety of different sensing technologies and algorithms are being implemented in devices for various biometric and biomedical applications, including health and wellness monitoring. This push is partly a result of the limitations in the usability of traditional measuring devices for continuous, noninvasive and/or ambulatory monitoring. Some such devices are, or include, photoacoustic sensors. Although some previously deployed devices can provide acceptable results, improved detection devices and systems would be desirable.

### SUMMARY

The systems, methods and devices of this disclosure each have several aspects, no single one of which is solely responsible for the desirable attributes disclosed herein.

In one aspect of the present disclosure, a method of determining a physiological parameter of a user is disclosed. In some embodiments, the method may include transmitting a continuous optical signal toward a skin of the user, the skin being proximate to a blood vessel of the user; during the transmitting of the continuous optical signal, causing generation of one or more acoustic signals from a blood vessel of the user by transmitting one or more pulsed optical signals toward the blood vessel; during the generation of the one or more acoustic signals, detecting a reflection of the continuous optical signal from the skin of the user; and based on the detected reflection of the continuous optical signal, determining a physiological parameter of the user.

In another aspect of the present disclosure, a user device is disclosed. In some embodiments, the user device may include a first light source system configured to transmit one or more first optical signals toward the target object, the one or more second optical signals configured to generate one or more acoustic signals from the a target object; a second light source system configured to transmit a second optical signal toward a skin of the user, the skin being proximate to the target object of the user; a receiver configured to detect a reflection of the second optical signal from the skin of the user during the generation of the one or more acoustic signals, the reflection of the second optical signal indicative of a physiological parameter of the user; and a wearable structure securable to the user and including the first light source system, the second light source system, and the receiver.

In another aspect of the present disclosure, a non-transitory computer-readable apparatus is disclosed. In some embodiments, the non-transitory computer-readable apparatus may include a storage medium, the storage medium including a plurality of instructions configured to, when executed by a control system, cause an apparatus to transmit a continuous optical signal toward a skin of the user, the skin being proximate to a blood vessel of the user; during the transmitting of the continuous optical signal, cause generation of one or more acoustic signals from a blood vessel of the user by transmitting one or more pulsed optical signals toward the blood vessel; during the generation of the one or more acoustic signals, detect a reflection of the continuous optical signal from the skin of the user; and based on the detected reflection of the continuous optical signal, determine a physiological parameter of the user.

Details of one or more implementations of the subject matter described in this disclosure are set forth in the accompanying drawings and the description below. Other features, aspects, and advantages will become apparent from the description, the drawings and the claims. Note that the relative dimensions of the following figures may not be drawn to scale.

### BRIEF DESCRIPTION OF THE DRAWINGS

FIG. 1 shows an example of a blood pressure monitoring device based on photoacoustic plethysmography, which may be referred to herein as PAPG.

FIG. 2 is a block diagram that shows example components of a sensor apparatus according to some disclosed implementations.

FIGS. 3A and 3B illustrate example apparatus for determining different levels of displacement of an object using a focus differential.

FIGS. 4A-4C illustrate example detections of displacements of an object based on an astigmatic focus differential using an example apparatus.

FIGS. 5A and 5B illustrate example scenarios of constructive and destructive interference of reference and measurement optical signals.

FIG. 6 illustrates an example apparatus configured to utilize laser interferometry.

FIG. 7 illustrates an example apparatus for determining different levels of displacement of an object using a laser Doppler vibrometer (LDV).

FIG. 8 shows examples of heart rate waveform (HRW) features that may be extracted according to some implementations.

FIG. 9A shows an example monitoring device designed to be worn around a wrist according to some implementations.

FIG. 9B shows an example monitoring device designed to be worn on a finger according to some implementations.

FIG. 9C shows an example monitoring device designed to reside on an earbud according to some implementations.

FIG. 10 illustrates an example target object of a user and profiles associated with a pressure wave experienced by the example target object.

FIGS. 11A-11D illustrate views of an example configuration of a multi-light source apparatus at different times of operation, according to some embodiments.

FIG. 12 depicts an example waveform representing reflected optical signals measured by a receiver over time.

FIGS. 13A and 13B show example layouts of sensors of multi-light source apparatus.

FIGS. 14A and 14B depict simplified diagrams of example beamformed photoacoustic images based on an ultrasonic image of a blood vessel, which may be obtained according to some embodiments.

FIG. 15 is a flow diagram of a method of determining a physiological parameter of a user, according to some disclosed implementations.

Like reference numbers and designations in the various drawings indicate like elements.

### DETAILED DESCRIPTION

The following description is directed to certain implementations for the purposes of describing various aspects of this disclosure. However, a person having ordinary skill in the art will readily recognize that the teachings herein can be applied in a multitude of different ways. Some of the concepts and examples provided in this disclosure are especially applicable to blood pressure monitoring applications or monitoring of other physiological parameters, characteristics, or properties of a target object of a user, such as a blood vessel. However, some implementations also may be applicable to other types of biological sensing applications, as well as to other fluid flow systems. The described implementations may be implemented in any device, apparatus, or system that includes an apparatus as disclosed herein. In addition, it is contemplated that the described implementations may be included in or associated with a variety of electronic devices such as, but not limited to: mobile telephones, multimedia Internet enabled cellular telephones, mobile television receivers, wireless devices, smartphones, smart cards, wearable devices such as bracelets, armbands, wristbands, rings, headbands, patches, chest bands, anklets, etc., Bluetooth® devices, personal data assistants (PDAs), wireless electronic mail receivers, hand-held or portable computers, netbooks, notebooks, smartbooks, tablets, printers, copiers, scanners, facsimile devices, global positioning system (GPS) receivers/navigators, cameras, digital media players, game consoles, wrist watches, clocks, calculators, television monitors, flat panel displays, electronic reading devices (e.g., e-readers), mobile health devices, computer monitors, auto displays (including odometer and speedometer displays, etc.), cockpit controls and/or displays, camera view displays (such as the display of a rear view camera in a vehicle), architectural structures, microwaves, refrigerators, stereo systems, cassette recorders or players, DVD players, CD players, VCRs, radios, portable memory chips, washers, dryers, washer/dryers, parking meters, automobile doors, Internet of Things (IoT) devices, etc. Thus, the teachings are not intended to be limited to the specific implementations depicted and described with reference to the drawings; rather, the teachings have wide applicability as will be readily apparent to persons having ordinary skill in the art.

There is a strong need for accurate, non-invasive, continuous monitoring devices for both clinical and consumer applications, e.g., for measuring physiological parameters such as blood pressure of a user. In particular, non-invasive, contactless monitoring of blood pressure using a user-wearable device is desirable. Continuous blood pressure monitoring opens avenues for efficient and effective diagnosis and treatment of cardiovascular conditions (e.g., hypertension), cardiovascular event detection, and stress monitoring. It would also allow daily spot checks of cardiovascular conditions including blood pressure, as well as overnight sleep monitoring. Positive user experience during overnight sleep monitoring is desirable. For example, there should be minimal discomfort to the user during operation of the wearable device, including during sleep. Hence, a device that does not apply external pressure to the user is desirable.

In some approaches, the size or diameter (or other dimensions) of a blood vessel as blood flows through it can be particularly relevant. As blood flows through the blood vessel, the resulting pressure expands the walls of the blood vessel, causing strain or distention during each pulse. Measuring arterial parameters, such as the size or diameter of the blood vessel over time, as well as corresponding distention or strain and heart rate waveforms, can provide relevant information for estimating blood pressure. Additionally, pulse wave velocity (PWV)—the velocity of the pressure wave along the arterial walls—is another important characteristic for determining pressure. PWV is a function of the arterial wall stiffness and tension, blood density, body posture, blood pressure, and more. It would thus be valuable for blood pressure estimation to obtain such information with accuracy and convenience.

Contactless sensing mechanisms that allow collection of biometrics and measurement of physiological characteristics such as pulse wave velocity (PWV) of a blood vessel, and arterial measurements such as diameter, cross-sectional area, volume, and/or distension, could enable the above approaches. In some disclosed configurations, a sensing apparatus may include two types of light source. More specifically, a first light source may emit a continuous optical signal with a first wavelength. A second light source may emit pulsed optical signals with a second wavelength so as to obtain a photoacoustic response from the blood vessel. As acoustic waves generated from the pulsed signals cause motion in the blood vessel and surrounding tissue, reflections of the continuous optical signal from the surface of the skin can be detected by a receiver. The reflected signals (e.g., amplitudes of a waveform of the signals or an image generated from the signals) can correlate to the displacements of the skin surface, and arterial dimensions and distension, and thus, physical characteristic such as PWV. Physiological parameters such as blood pressure can then be derived.

Further, in some implementations, machine learning can be used to train a machine learning or artificial intelligence model that can predict a physiological characteristic (e.g., PWV) or parameter of the user (e.g., blood pressure). In addition, based on any discrepancies between the sensor-based estimation and the model-generated prediction, some or all of the sensor-based measurements can be kept or discarded.

Particular implementations of the subject matter described in this disclosure can be implemented to realize one or more of the following potential advantages. Only using optical sensing systems and detection techniques eliminates the requirement of a direct coupling medium such as a gel. They further eliminate the need to apply an external counterpressure that can change the dimensions of the blood vessel, which can complicate measurements and cause user discomfort. Measurements with counterpressure applied are not the same as an undisturbed blood vessel. Complex hardware is not needed to implement the optical sensing systems. Physiological characteristics (e.g., PWV) can be derived to accurately estimate a difficult physiological parameter to obtain such as blood pressure in a simplified way. The signal data obtained may also be compatible with machine learning or deep learning implementations, where the data can be used as input to a machine learning or artificial intelligence model and further improve the accuracy of the estimated blood pressure.

Additional details will follow after an initial description of relevant systems and technologies.

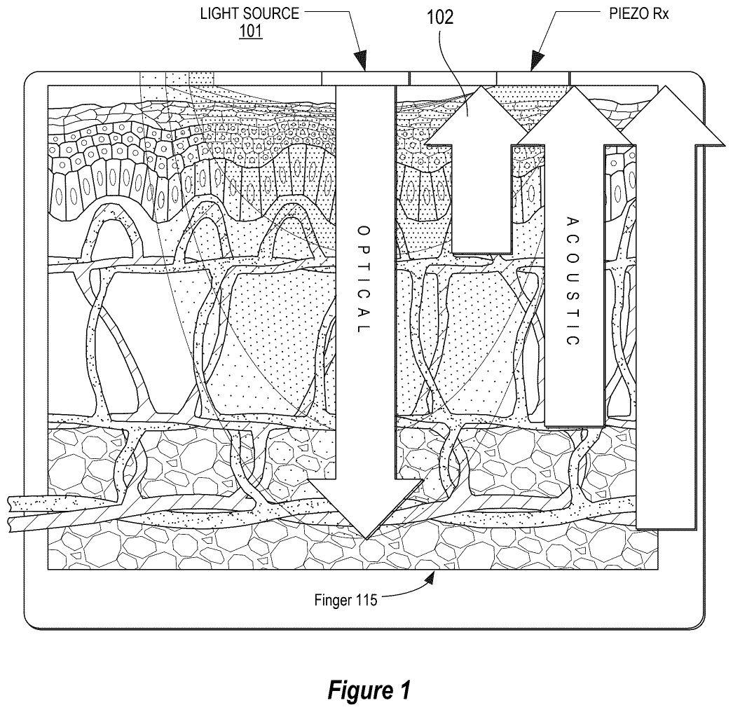

FIG. 1 shows an example of a blood pressure monitoring device based on photoacoustic plethysmography, which is referred to herein as PAPG. FIG. 1 shows the same examples of arteries, veins, arterioles, venules and capillaries inside a body part, which is a finger 115 in this example. In some examples, the light source 101 shown in FIG. 1 may be coupled to a light source system (not shown) that is disposed remotely from the body part (e.g., finger 115). In some implementations, the light source 101 may be an opening of an optical fiber or other waveguide. Such an opening may also be connected to an opening of an interface that is contactable with the body part. In some embodiments, the light source system may include one or more LEDs, one or more laser diodes, etc. In this example, the light source 101 has transmitted light (in some examples, green, red, infrared, and/or near-infrared (NIR) light) that has penetrated the tissues of the finger 115 in an illuminated zone.

In the example shown in FIG. 1, blood vessels (and components of the blood itself) are heated by the incident light from the light source 101 and are emitting acoustic waves 102. In this example, the emitted acoustic waves 102 include ultrasonic waves. According to this implementation, the acoustic wave emissions 102 are being detected by an ultrasonic receiver, which is a piezoelectric receiver in this example. Photoacoustic emissions 102 from the illuminated tissues, detected by the piezoelectric receiver, may be used to detect volumetric changes in the blood of the illuminated zone of the finger 115 that correspond to physiological data within the illuminated tissues of finger 115, such as heart rate waveforms. Although some of the tissue areas shown to be illuminated are offset from those shown to be producing photoacoustic emissions 102, this is merely for illustrative convenience. It will be appreciated that that the illuminated tissues will actually be those producing photoacoustic emissions. Moreover, it will be appreciated that the maximum levels of photoacoustic emissions will often be produced along the same axis as the maximum levels of illumination.

One important difference between an optical technique such as a photoplethysmography (PPG)-based system the PAPG-based method of FIG. 1 is that the acoustic waves shown in FIG. 1 travel much more slowly than the reflected light waves involved in PPG. Accordingly, depth discrimination based on the arrival times of the acoustic waves shown in FIG. 1 is possible, whereas depth discrimination based on the arrival times of the light waves in PPG may not be possible. This depth discrimination allows some disclosed implementations to isolate acoustic waves received from the different blood vessels.

According to some such examples, such depth discrimination allows artery heart rate waveforms to be distinguished from vein heart rate waveforms and other heart rate waveforms. Therefore, blood pressure estimation based on depth-discriminated PAPG methods can be substantially more accurate than blood pressure estimation based on PPG-based methods.

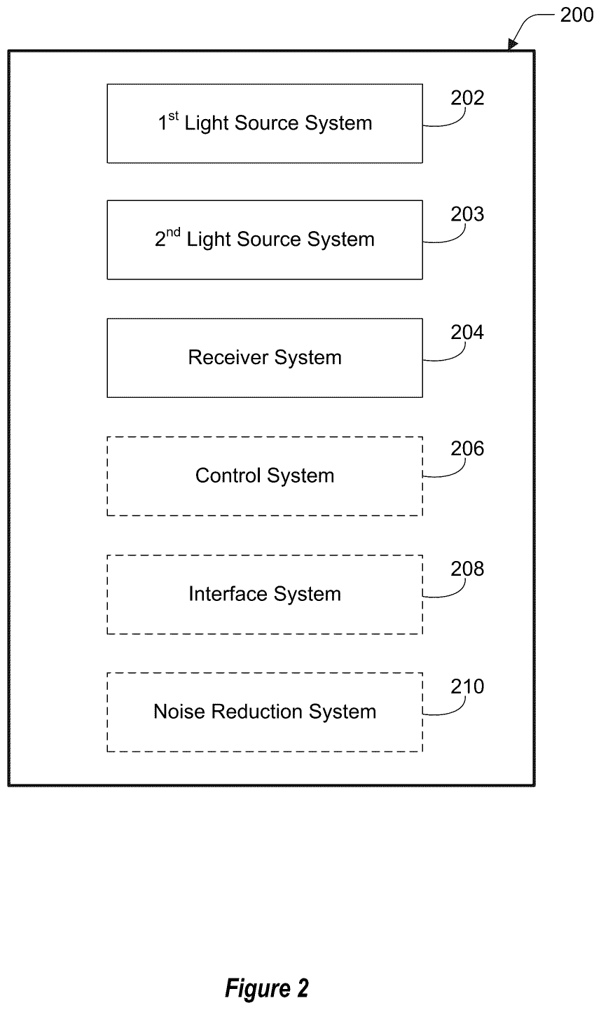

FIG. 2 is a block diagram that shows example components of a sensor apparatus 200 according to some implementations. In this example, the sensor apparatus 200 may include a first light source system 202, a second light source system 203, and a receiver system 204. Some implementations of the sensor apparatus 200 may include a control system 206, an interface system 208, a noise reduction system 210, or a combination thereof.

Various configurations of first light source system 202, second light source system 203, and receiver system 204 are disclosed herein. Specific examples are described in more detail below.

In some embodiments, the first light source system 202 may be an example of a light source system that is coupled to a light source 101 as shown in FIG. 1. That is to say, in some implementations, the first light source system 202 may be configured to generate optical signals and trigger a photoacoustic (PAPG) response from a target object, such as a blood vessel or other tissue, and cause emission of acoustic waves (e.g., acoustic waves 102 as illustrated in FIG. 1).

According to some embodiments, the first light source system 202 may include a light source configured to produce and direct light. In some cases, the first light source system 202 may include one or more one or more light sources. In some implementations, the first light source system 202 may include one or more light-emitting diodes (LEDs). In some implementations, the first light source system 202 may include one or more laser diodes. According to some implementations, the first light source system 202 may include one or more vertical-cavity surface-emitting lasers (VCSELs). In some implementations, the first light source system 202 may include one or more edge-emitting lasers. In some implementations, the first light source system 202 may include one or more neodymium-doped yttrium aluminum garnet (Nd:YAG) lasers. In some implementations, the first light source system 202 may include at least one multi-junction laser diode, which may produce less noise than single-junction laser diodes.

Hence, the first light source system 202 may include, for example, a laser diode, a light-emitting diode (LED), or a line or an array of either or both. In addition, in some implementations, the light source (e.g., laser diode, LED) of the first light source system 202 may be steered to different directions and locations. A line or an array of light sources of the first light source system 202 can similarly be steered individually or collectively. The first light source system 202 may be configured to, via the laser diode(s) or LEDs, generate and emit optical signals. The first light source system 202 may, in some examples, be configured to transmit light or optical signals in one or more wavelength ranges. An example wavelength range for the first light source system 202 may be 400 to 1200 nanometers (nm). Some applications may use higher wavelengths, as noted below. In some examples, the first light source system 202 may be configured to transmit light in a wavelength range of 500 to 600 nm. According to some examples, the first light source system 202 may be configured to transmit light in a wavelength range of 800 to 950 nm. According to some examples, the first light source system 202 may be configured to transmit light in infrared or near infrared (NIR) region of the electromagnetic spectrum (about 700 to 2500 nm). In view of factors such as skin reflectance, fluence, the absorption coefficients of blood and various tissues, and skin safety limits, one or both of these wavelength ranges may be suitable for various use cases. For example, the wavelength ranges of 500 nm to 600 nm and of 800 to 950 nm may both be suitable for obtaining photoacoustic responses from relatively smaller, shallower blood vessels, such as blood vessels having diameters of approximately 0.5 mm and depths in the range of 0.5 mm to 1.5 mm, such as may be found in a finger. The wavelength range of 800 to 950 nm, or about 700 to 900 nm, or about 600 to 1100 nm may, for example, be suitable for obtaining photoacoustic responses from relatively larger, deeper blood vessels, such as blood vessels having diameters of approximately 2.0 mm and depths in the range of 2 mm to 3 mm, such as may be found in an adult wrist. In some implementations, the first light source system 202 may be configured to switch wavelengths to capture acoustic information from different depths, e.g., based on signal(s) from the control system 206.

In some implementations, the first light source system 202 may be configured for emitting one or more wavelengths of light, which may be selectable to trigger acoustic wave emissions primarily from a particular type of material. For example, because the hemoglobin in blood absorbs near-infrared light very strongly, in some implementations, the first light source system 202 may be configured for emitting one or more wavelengths of light in the near-infrared range, in order to trigger acoustic wave emissions from hemoglobin. However, in some examples, the control system 206 may control the wavelength(s) of light emitted by the first light source system 202 to preferentially induce acoustic waves in blood vessels, other soft tissue, and/or bones. For example, an infrared (IR) light-emitting diode LED may be selected and a short pulse of IR light emitted to illuminate a portion of a target object and generate acoustic wave emissions that are then detected by the receiver system 204. In some implementations, the first light source system 202 may be configured to select specific wavelength values, such as 808 nm, 905 nm, or 940 nm. In another example, an IR LED and a red LED or other color such as green, blue, white or ultraviolet (UV) may be selected and a short pulse of light emitted from each light source in turn with ultrasonic images obtained after light has been emitted from each light source. In other implementations, one or more light sources of different wavelengths may be fired in turn or simultaneously to generate acoustic emissions that may be detected by an ultrasonic receiver of the receiver system 204. Image data from the ultrasonic receiver that is obtained with light sources of different wavelengths and at different depths (e.g., varying range gate delays (RGDs)) into the target object may be combined to determine the location and type of material in the target object. Image contrast may occur as materials in the body generally absorb light at different wavelengths differently. As materials in the body absorb light at a specific wavelength, they may heat differentially and generate acoustic wave emissions with sufficiently short pulses of light having sufficient intensities. Depth contrast may be obtained with light of different wavelengths and/or intensities at each selected wavelength. That is, successive images may be obtained at a fixed RGD (which may correspond with a fixed depth into the target object) with varying light intensities and wavelengths to detect materials and their locations within a target object. For example, hemoglobin, blood glucose or blood oxygen within a blood vessel inside a target object such as a finger may be detected photoacoustically.

In various implementations, the first light source system 202 may be configured to emit pulses of light or optical signals having a pulse width. According to some implementations, the first light source system 202 may be configured for emitting a light pulse with a pulse width less than about 100 nanoseconds. In some implementations, the light pulse may have a pulse width between about 10 nanoseconds and about 500 nanoseconds or more (e.g., from 3 nanoseconds to 1000 nanoseconds). In some cases, the pulse width may be selected from a range between about 50 nanoseconds and about 200 nanoseconds. According to some examples, the first light source system 202 may be configured for emitting a plurality of light pulses at a pulse repetition frequency between 10 Hz and 100 kHz, or in some cases, between 50 Hz and 25 kHz, or between 1 kHz and 5 kHz. Alternatively, or additionally, in some implementations the first light source system 202 may be configured for emitting a plurality of light pulses at a pulse repetition frequency between about 1 MHz and about 100 MHz. Alternatively, or additionally, in some implementations, the first light source system 202 may be configured for emitting a plurality of light pulses at a pulse repetition frequency between about 10 Hz and about 1 MHz. In some examples, the pulse repetition frequency of the light pulses may correspond to an acoustic resonant frequency of the ultrasonic receiver and/or other parts of the sensor apparatus 200. For example, a set of four or more light pulses may be emitted from the first light source system 202 at a frequency that corresponds with the resonant frequency of a resonant acoustic cavity in the sensor stack, allowing a build-up of the received ultrasonic waves and a higher resultant signal strength. In some implementations, filtered light or light sources with specific wavelengths for detecting selected materials may be included with the first light source system 202. In some implementations, the first light source system 202 may contain light sources such as red, green and blue LEDs of a display that may be augmented with light sources of other wavelengths (such as IR and/or UV) and with light sources of higher optical power. For example, high-power laser diodes or electronic flash units (e.g., an LED or xenon flash unit) with or without filters may be used for short-term illumination of the target object.

According to some examples, the first light source system 202 may also include one or more light-directing elements configured to direct light from the first light source system 202 towards the target object. In some examples, the one or more light-directing elements may include at least one diffraction grating. Alternatively, or additionally, the one or more light-directing elements may include at least one lens. In some implementations, the first light source system 202 may be configured to direct light using one or more optical waveguides (e.g., optical fibers) configured to direct light toward the target object.

In various configurations, the first light source system 202 may incorporate anti-reflection (AR) coating, a mirror, a light-blocking layer, a shield to minimize crosstalk, etc.

The first light source system 202 may include various types of drive circuitry, depending on the particular implementation. In some examples, the first light source system 202 may include a drive circuit (also referred to herein as drive circuitry) configured to cause the first light source system 202 to emit pulses of light at pulse widths in a range from 3 nanoseconds to 1000 nanoseconds. According to some examples, the first light source system 202 may include a drive circuit configured to cause the light source system 204 to emit pulses of light at pulse repetition frequencies in a range from 1 kilohertz to 100 kilohertz.

In some example implementations, some or all of the one or more light sources of the first light source system 202 may be disposed at or along an axis that is parallel to or angled relative to a central axis associated with the sensor apparatus 200. Optical signals may be emitted toward a target object (e.g., blood vessel), which may cause generation of ultrasonic waves by the target object. These ultrasonic waves may be detectable by one or more receiver elements of a receiver system 204.

In some embodiments, the second light source system 203 may be configured to generate optical signals having one or more wavelengths of light. In some implementations, the second light source system 203 may include one or more LEDs. In some implementations, the second light source system 203 may include one or more laser diodes. According to some implementations, the second light source system 203 may include one or more VCSELs. In some implementations, the second light source system 203 may include one or more edge-emitting lasers. In some implementations, the second light source system 203 may include one or more Nd:YAG lasers. In some implementations, the second light source system 203 may include at least one multi-junction laser diode, which may produce less noise than single-junction laser diodes. Hence, the second light source system 203 may include, for example, a laser diode, an LED, or a line or an array of either or both. In addition, in some implementations, the light source (e.g., laser diode, LED) of the second light source system 203 may be steered to different directions and locations. A line or an array of light sources of the second light source system 203 can similarly be steered individually or collectively. The second light source system 203 may be configured to, via the laser diode(s) or LEDs, generate and emit optical signals. The second light source system 203 may, in some examples, be configured to transmit light or optical signals in one or more wavelength ranges.

In some examples, the control system 206 may control the wavelength(s) of light emitted by the second light source system 203. The second light source system 203 may, in some examples, be configured to transmit light in a wavelength range of about 500 nm to 16000 nm (16 micrometers (μm)). In some examples, the second light source system 203 may be configured to transmit light in a wavelength range of 500 nm to 700 nm. In some examples, the second light source system 203 may be configured to transmit light in a wavelength of 1550 nm.

In some implementations, the optical signals emitted by the second light source system 203 may be continuous or relatively continuous, rather than in short pulses. The continuous optical signal emitted by the second light source system 203 may last for up to 10 microseconds (or more). In some cases, the optical signals emitted by the second light source system 203 may be characterized as a longer pulse (e.g., 10 microseconds or more) than that emitted by the first light source system 202 (e.g., 100 nanoseconds at most). The first light source system 202 may be configured to emit pulses of optical signals during this continuous or longer pulse time, at example pulse widths and example pulse repetition frequencies specified according to the above.

In contrast to the first light source system 202, the wavelength selected for the second light source system 203 may not be configured to trigger a photoacoustic (PAPG) response from the target object. Moreover, the wavelength(s) selected for the first light source system 202 and the wavelength(s) selected for the second light source system 203 may be substantially distinct from one another to prevent interference between optical signals emitted by the first light source system 202 and optical signals emitted by the second light source system 203. For example, the difference in wavelengths between the optical signals emitted by the first light source system 202 and those emitted by the second light source system 203 may be selected to be between at least about 3 to 50 nm. An example pair of wavelengths for respective optical signals emitted by the first light source system 202 and the second light source system 203 may be 808 nm (for the first light source system 202) and 1550 nm (for the second light source system 203). Other example pairs of wavelengths may be 498 nm, 568 nm, or 794 nm for the first light source system 202 for generating a photoacoustic response, and 632 nm or 1152 nm for the second light source system 203 for continuous light.

Various examples of a receiver system 204 are disclosed herein, some of which may include ultrasonic receiver systems, optical receiver systems, or combinations thereof. In some implementations, the receiver system 204 includes an ultrasonic receiver system having the one or more receiver elements. In implementations that include an ultrasonic receiver system, the ultrasonic receiver and an ultrasonic transmitter may be combined in an ultrasonic transceiver. In some examples, the receiver system 204 may include a piezoelectric receiver layer, such as a layer of PVDF polymer or a layer of PVDF-TrFE copolymer. In some implementations, a single piezoelectric layer may serve as an ultrasonic receiver. In some implementations, other piezoelectric materials may be used in the piezoelectric layer, such as aluminum nitride (AlN) or lead zirconate titanate (PZT). The receiver system 204 may, in some examples, include an array of ultrasonic transducer elements, such as an array of piezoelectric micromachined ultrasonic transducers (PMUTs), an array of capacitive micromachined ultrasonic transducers (CMUTs), etc. In some such examples, a piezoelectric receiver layer, PMUT elements in a single-layer array of PMUTs, or CMUT elements in a single-layer array of CMUTs, may be used as ultrasonic transmitters as well as ultrasonic receivers. According to some examples, the receiver system 204 may be, or may include, an ultrasonic receiver array. In some examples, the sensor apparatus 200 may include one or more separate ultrasonic transmitter elements or one or more separate arrays of ultrasonic transmitter elements. In some examples, the ultrasonic transmitter(s) may include an ultrasonic plane-wave generator.

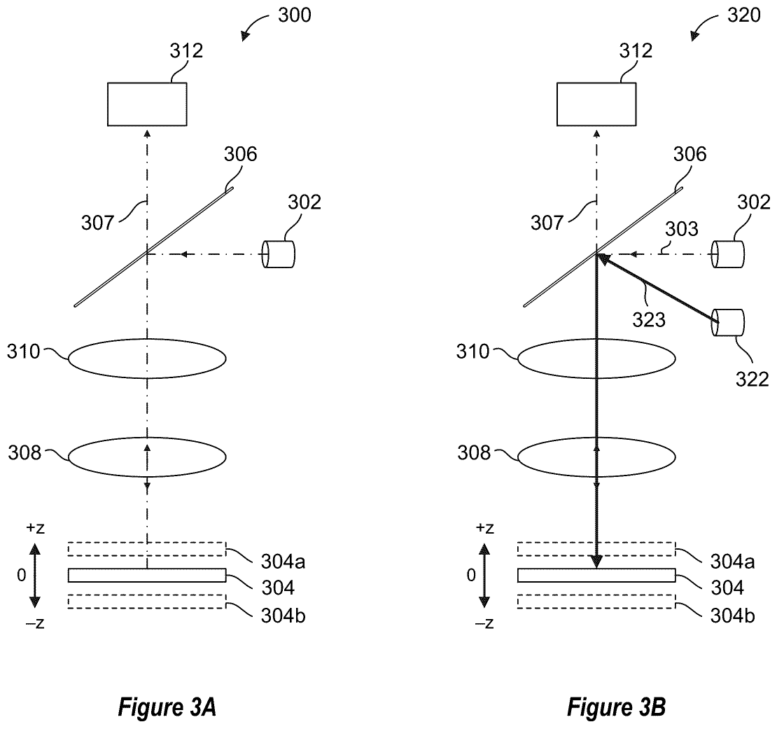

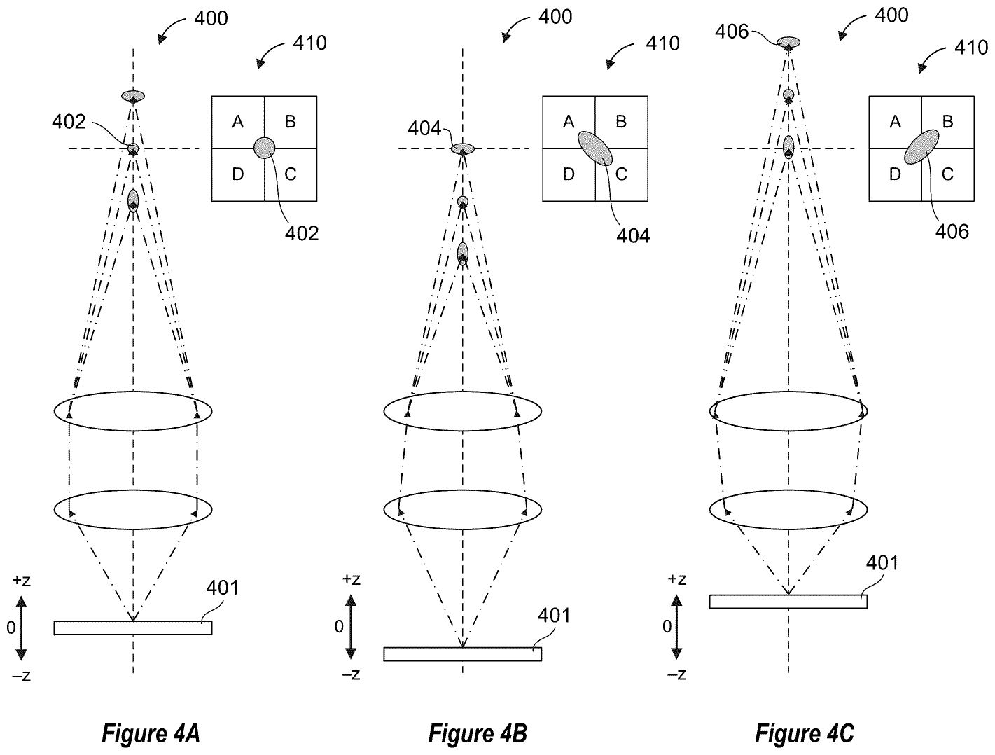

In some embodiments, the receiver system 204 may include an optical detector (e.g., a photodetector or photosensor) that is part of an optical pick up (OPU). An OPU may refer to a sensing apparatus that measures vibrations or motion of a surface. In some examples disclosed herein, principles of a focus differential may be used to determine the motion of the surface of a user's skin. In some implementations, the second light source system 203 and the receiver system 204 may form the OPU. In some examples, optical signals may be directed toward the user's skin from the second light source system 203, and optical signals reflected from the skin may be detected by the photodetector of the receiver system 204 which may be configured to determine signal characteristics such as amplitude or frequency. In some implementations, the second light source system 203 may include a laser that emits a continuous optical signal. The receiver system 204 may be configured to obtain a continuous waveform (e.g., of continuous analog voltage) corresponding to the reflection of the continuous optical signal, from which amplitude and/or frequency may be determined. In some cases where non-continuous light is emitted or detected, discrete values of amplitude and/or frequency may be determined. Aforementioned principles of the focus differential are described in further detail in FIGS. 3A-4C.

One illustrative example of a cycle of pulses within a period of continuous optical signal may be a plurality of pulses from the first light source system 202 lasting 200 ns each within a period of 10 microseconds during which the continuous optical signal is emitted from the second light source system 203 and reflected toward the receiver system 204.

FIG. 3A illustrates an example apparatus 300 for determining different levels of displacement of an object using a focus differential. In some configurations, the example apparatus 300 may include a light source 302, which may be an example of the second light source system 203 or a portion thereof, which may emit light 303 that reflects from a surface 304. In some applications, the surface 304 may be the surface of skin, and light 303 may not trigger a photoacoustic response from a target object underneath the skin (such as from a blood vessel). That is, the wavelength of light 303 may not cause the photoacoustic response.

The example apparatus 300 may further include an objective lens 308, a collimating lens 310, and/or other lenses such as a cylindrical lens. In some cases, the light may reflect from a splitter 306 (e.g., a polarizing beam splitter) that allows reflected light 307 to pass through. The example apparatus 300 may further include a photosensor 312, which may be configured to detect reflected light. The photosensor 312 may be an example of the receiver system 204 or a portion thereof.

FIG. 3B illustrates another example apparatus 320 for determining different levels of displacement of an object using a focus differential. In this example, another light source 322 may be included, which may be an example of the first light source system 202 or a portion thereof, which may emit light 323. In some applications, light 323 may be configured to trigger a photoacoustic response from a target object. In other words, the wavelength of light 323 may be such that it causes the photoacoustic response. In some configurations, photosensor 312 may not detect any reflections of light 323, or may ignore or discard any detections. Moreover, light 303 and light 323 may be emitted concurrently; e.g., light 323 may be emitted as pulses during continuous emission of light 303. In some implementations, the wavelength of light 303 and light 323 may be sufficiently distinct to prevent interference (e.g., by a difference of at least about 3-50 nm or beyond a threshold of 50 nm). Although not shown, other paths of light may be possible for both the light source 302 and the light source 322.

Depending on the scenario, the surface 304 may be displaced into a different plane at a different time, moved above or below relative to a z-axis, e.g., caused by vibration or other motion. For example, surface 304a may be displaced above (+z) the neutral plane (z=0), and surface 304b may be displaced below (−z) the neutral plane. The detection and measurement of the displacement will now be described with examples.

FIG. 4A illustrates an example detection of a displacement of an object 401 based on an astigmatic focus differential using an example apparatus 400. The apparatus 400 may include a photosensor 410. In some implementations, the apparatus 400 may be an example of the example apparatus 300 or 320, and the photosensor 410 may be an example of the photosensor 312.

In some configurations, the photosensor 410 may include at least four detectors A, B, C, D arranged in quadrants as shown. Each detector may be configured to detect intensity of light, which may be expressed as a voltage. Voltage from detector A may be expressed as V_{A}, voltage from detector B may be expressed as V_{B}, voltage from detector B may be expressed as V_{C}, and voltage from detector B may be expressed as V_{D}. A focus error may occur when the optical path possesses asymmetry. This optical asymmetry in an optical system may be referred to as astigmatism and can be determined from the expression (V_{A}+V_{C})−(V_{B}+V_{D}).

In the FIG. 4A example, the resulting signal 402 from the expression (V_{A}+V_{C})−(V_{B}+V_{D}) is equal to 0. This can signify that there is no focus error and that the object 401 is in focus. There is no displacement of the object 401 detected with respect to its neutral plane (z=0).

FIG. 4B illustrates another example detection of a displacement of the object 401. In this example, the resulting signal 404 from the expression (V_{A}+V_{C})−(V_{B}+V_{D}) is greater than 0. This can signify that the object 401 is too far away (or farther from the photosensor 410 than the neutral plane), displaced below (−z) the neutral plane, and not in focus. FIG. 4C illustrates another example detection of a displacement of the object 401. In this example, the resulting signal 404 from the expression (V_{A}+V_{C})−(V_{B}+V_{D}) is less than 0. This can signify that the object 401 is too close (or closer to the photosensor 410 than the neural plane), displaced above (+z) the neutral plane, and not in focus. The amount of displacement is proportional to the magnitude of the resulting signal. As such, the receiver system 204 may be capable of determining a displacement or distance to a reference location (e.g., surface of skin) over time.

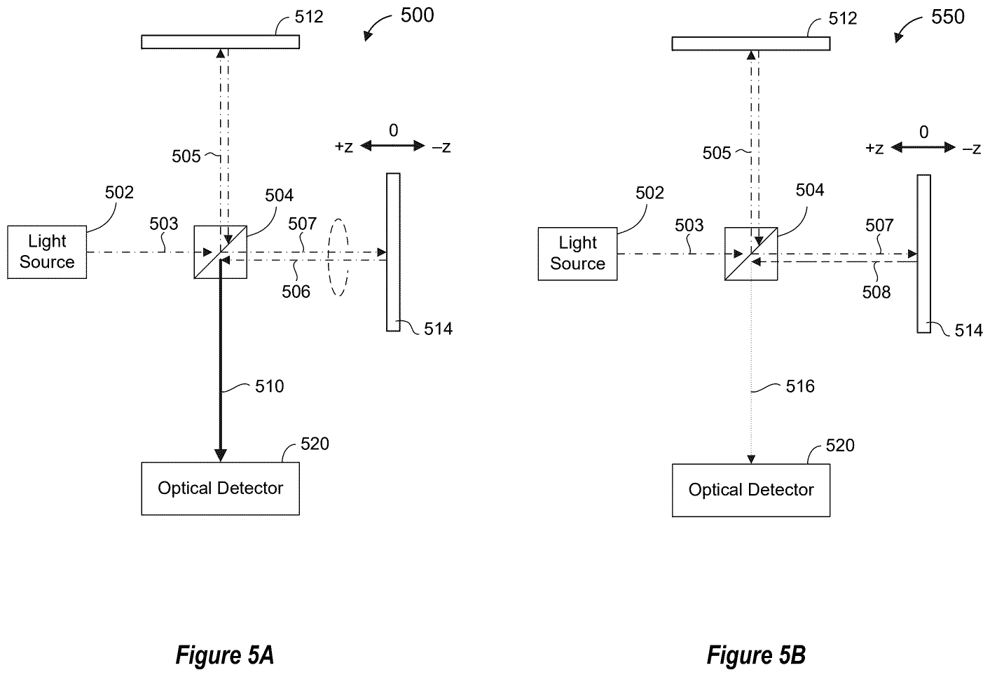

In some configurations, principles of interferometry can be useful for detecting motion of a surface of an object. FIG. 5A shows an example scenario 500 of constructive interference of a reference optical signal 505 and a measurement optical signal 507. A light source 502 may generate and emit an optical signal 503 such as a laser beam. Light source 502 may be an example of the second light source system 203 or a portion thereof. The optical signal 503 may be split into a reference optical signal 505 and a measurement optical signal 507 (each having approximately half signal intensity) using a splitter 504. The reference optical signal 505 may be reflected from a stationary surface such as a stationary mirror 512 and directed back toward the splitter 504, while the measurement optical signal 507 may be reflected from a moving surface such as a moving mirror 514 and directed back toward the light source 502. Reflected optical signal 506 may be “in phase” with the reference optical signal 505. In this example, they may be exactly in phase, which may result in a strong combined optical signal 510 and detected by an optical detector 520 (e.g., a photodetector or photosensor). Optical detector 520 may be an example of the receiver system 204 or a portion thereof.

FIG. 5B shows an example scenario 550 of constructive interference of a reference optical signal 505 and a measurement optical signal 507. Here, reflected optical signal 508 from the moving mirror 514 may have a phase difference or shift that is above 0 degree as a result of the distance of the moving mirror 514 relative to its non-moving (0) position. The phase shift may be proportional to the distance of the moving mirror 514, and hence can be a proxy for the position of a moving surface. There will be destructive interference, and a resulting combined optical signal 516 at the optical detector 520 may be weak or close to zero in the case of a phase that is close to 180 degrees.

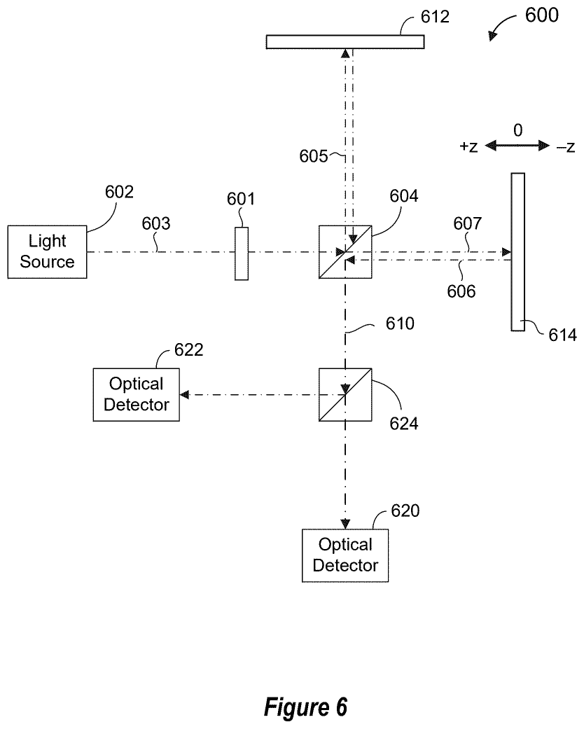

FIG. 6 illustrates an example apparatus configured to utilize laser interferometry. Similar to the scenarios shown in FIGS. 5A and 5B, a light source 602 may emit an optical signal 603, which may be split into a reference optical signal 605 and a measurement optical signal 607 using a splitter 604. Light source 602 may be an example of the second light source system 203 or a portion thereof. In addition, optical signal 603 may pass through a polarizer 601, resulting in the optical signal 603 becoming circularly polarized (having orthogonal fields that are 90 degrees apart in phase). A polarizing splitter 624 may split the optical signal 610 into two orthogonal fields, which may be detected by a first optical detector 620 and a second optical detector 622. Depending on the direction of the motion of a moving surface (e.g., moving mirror 614), the first optical detector 620 may lead or lag the second optical detector 622, which may be indicative of a phase difference between the split optical signals. First optical detector 620 and/or second optical detector 622 may be example(s) of the receiver system 204 or a portion thereof. This phase information may be used to determine direction and thus the position of the moving surface.

In some embodiments, the receiver system 204 may include an optical detector (e.g., a photodetector or photosensor) that is part of a laser Doppler vibrometer (LDV). Such an optical detector may detect reflected optical signals (e.g., from a continuous laser), which may be generated and aimed at a surface (e.g., skin of a user). Based on such reflected optical signals, displacement and/or velocity of the surface along the direction of the laser is measured through the Doppler effect and the principles of interferometry. As is known, the Doppler effect is the apparent change or shift in frequency observed when the source of a wave is moving relative to the observer. This frequency shift is proportional to the velocity of relative movement between the observer and the wave source.

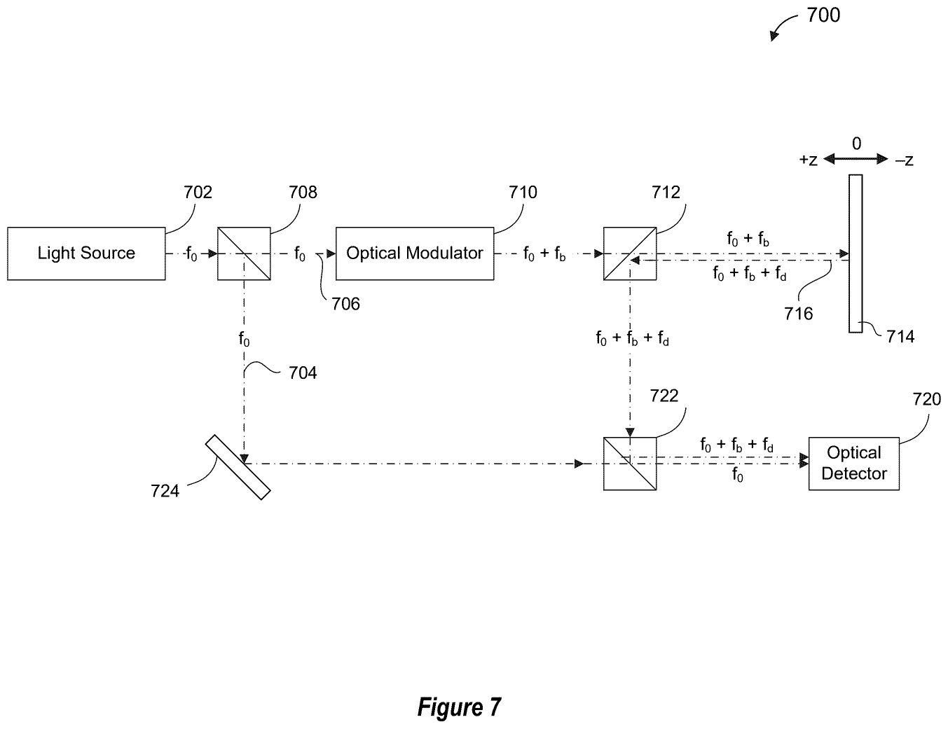

FIG. 7 illustrates an example apparatus 700 for determining different levels of displacement of an object an LDV. Within the example apparatus 700, an optical signal (having a frequency of f₀) such as a laser beam from a light source 702 may be split into a reference optical signal 704 and a measurement optical signal 706 using a splitter 708. Light source 702 may be an example of the second light source system 203 or a portion thereof. That is, a continuous (or relatively continuous) optical signal may be emitted to measure the displacement of an object or surface 714, e.g., caused by vibration. An optical modulator 710 may be used to shift the frequency of the reference optical signal 704 by f_{b} (e.g., 40 MHz). The optical modulator 710 may include an acousto-optic modulator such as a Bragg Cell, which may be configured to diffract and shift the frequency of input light using acoustic waves. A reflected optical signal 716, which has passed through another splitter 712 and reflected from a surface of an object (which may be vibrating or otherwise in motion), may have experienced a frequency shift of f_{d}, which is proportional to the velocity of the surface, resulting in a frequency of f₀+f_{b}+f_{d}. The reflected optical signal 716 may be guided to an optical detector 720 via a third splitter 722. The reference optical signal 704 reflected from the splitter 708 may also be guided to the optical detector 720, e.g., via a mirror 724. Hence, the optical detector 720 may receive two different optical signals having frequencies of f₀+f_{b}+f_{d} and f₀. Without the optical modulator 710, the optical detector 720 may not be able to distinguish whether the detected movement of the surface 714 is towards or away from the measurement optical signal 706. Since f_{d} is proportional to velocity, the displacement and/or velocity of the surface 714 may thereby be determined.

Therefore, various approaches may be taken to determine the motion of a surface such a user's skin, including its displacement and/or velocity. As discussed above, these approaches may include OPU, interferometry, and LDV.

Referring back to FIG. 2, in some implementations, at least portions of the sensor apparatus 200 (for example, the first light source system 202, the second light source system 203, the receiver system 204, or a combination thereof) may include one or more sound-absorbing layers, acoustic isolation material, light-absorbing material, light-reflecting material, or combinations thereof. In some examples, acoustic isolation material may reside between the first light source system 202 (and/or the second light source system 203) and at least a portion of the receiver system 204. In some examples, at least portions of the sensor apparatus 200 (for example, the first light source system 202, the second light source system 203, the receiver system 204, or a combination thereof) may include one or more electromagnetically shielded transmission wires. In some such examples, the one or more electromagnetically shielded transmission wires may be configured to reduce electromagnetic interference from the first light source system 202 (and/or the second light source system 203) that is received by the receiver system 204.

The control system 206 may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system 206 also may include (and/or be configured for communication with) one or more memory devices, such as one or more random access memory (RAM) devices, read-only memory (ROM) devices, etc. Accordingly, the sensor apparatus 200 may have a memory system that includes one or more memory devices, though the memory system is not shown in FIG. 2. The control system 206 may be configured for receiving and processing data from the receiver system 204, e.g., as described below. If the sensor apparatus 200 includes an ultrasonic transmitter, the control system 206 may be configured for controlling the ultrasonic transmitter. In some implementations, functionality of the control system 206 may be partitioned between one or more controllers or processors, such as a dedicated sensor controller and an applications processor of a mobile device.

In some examples, the control system 206 may be communicatively coupled to the first light source system 202 and the second light source system 203, and configured to control each light source system to emit light towards a target object. In some such examples, the control system 206 may be configured to receive signals from the receiver system 204 (including one or more receiver elements) corresponding to the ultrasonic waves generated by the target object responsive to the light from the light source system. In some examples, the control system 206 may be configured to identify one or more blood vessel signals, such as arterial signals or vein signals, from the ultrasonic receiver system. In some such examples, the one or more arterial signals or vein signals may be, or may include, one or more blood vessel wall signals corresponding to ultrasonic waves generated by one or more arterial walls or vein walls of the target object. In some such examples, the one or more arterial signals or vein signals may be, or may include, one or more arterial blood signals corresponding to ultrasonic waves generated by blood within an artery of the target object or one or more vein blood signals corresponding to ultrasonic waves generated by blood within a vein of the target object. In some examples, the control system 206 may be configured to determine or estimate one or more physiological parameters or cardiac features based, at least in part, on one or more arterial signals, on one or more vein signals, or on combinations thereof. According to some examples, a physiological parameter may be, or may include, blood pressure. In some approaches, blood pressure can be estimated based at least on PWV, as will be discussed below.

In further examples, the control system 206 may be communicatively coupled to the receiver system 204. The receiver system 204 may be configured to detect acoustic signals from the target object. The control system 206 may be configured to select at least one of a plurality of receiver elements of the receiver system 204. Such selected receiver element(s) may correspond to the best signals from multiple receiver elements. In some embodiments, the selection of the at least one receiver element may be based on information regarding detected acoustic signals (e.g., arterial signals or vein signals) from the plurality of receivers. For example, signal quality or signal strength (based, e.g., on signal-to-noise ratio (SNR)) of some signals may be relatively higher than some others or above a prescribed threshold or percentile, which may indicate the best signals. In some implementations, the control system 206 may also be configured to, based on the information regarding detected acoustic signals, determine or estimate at least one characteristic of the blood vessels such as PWV (indicative of arterial stiffness), arterial dimensions, or both.

Some implementations of the sensor apparatus 200 may include an interface system 208. In some examples, the interface system 208 may include a wireless interface system. In some implementations, the interface system 208 may include a user interface system, one or more network interfaces, one or more interfaces between the control system 206 and a memory system and/or one or more interfaces between the control system 206 and one or more external device interfaces (e.g., ports or applications processors), or combinations thereof. According to some examples in which the interface system 208 is present and includes a user interface system, the user interface system may include a microphone system, a loudspeaker system, a haptic feedback system, a voice command system, one or more displays, or combinations thereof. According to some examples, the interface system 208 may include a touch sensor system, a gesture sensor system, or a combination thereof. The touch sensor system (if present) may be, or may include, a resistive touch sensor system, a surface capacitive touch sensor system, a projected capacitive touch sensor system, a surface acoustic wave touch sensor system, an infrared touch sensor system, any other suitable type of touch sensor system, or combinations thereof.

In some examples, the interface system 208 may include a force sensor system. The force sensor system (if present) may be, or may include, a piezo-resistive sensor, a capacitive sensor, a thin film sensor (for example, a polymer-based thin film sensor), another type of suitable force sensor, or combinations thereof. If the force sensor system includes a piezo-resistive sensor, the piezo-resistive sensor may include silicon, metal, polysilicon, glass, or combinations thereof. An ultrasonic fingerprint sensor and a force sensor system may, in some implementations, be mechanically coupled. In some implementations, the force sensor system may be mechanically coupled to a platen. In some such examples, the force sensor system may be integrated into circuitry of the ultrasonic fingerprint sensor. In some examples, the interface system 208 may include an optical sensor system, one or more cameras, or a combination thereof.

According to some examples, the sensor apparatus 200 may include a noise reduction system 210. For example, the noise reduction system 210 may include one or more mirrors that are configured to reflect light from the first light source system 202 and/or the second light source system 203 away from the receiver system 204. In some implementations, the noise reduction system 210 may include one or more sound-absorbing layers, acoustic isolation material, light-absorbing material, light-reflecting material, or combinations thereof. In some examples, the noise reduction system 210 may include acoustic isolation material, which may reside between the first light source system 202 (and/or the second light source system 203) and at least a portion of the receiver system 204, on at least a portion of the receiver system 204, or combinations thereof. In some examples, the noise reduction system 210 may include one or more electromagnetically shielded transmission wires. In some such examples, the one or more electromagnetically shielded transmission wires may be configured to reduce electromagnetic interference from circuitry of the light source system, receiver system circuitry, or combinations thereof, that is received by the receiver system.

In some configurations, the first light source system 202, second light source system 203, and receiver system 204 may be implemented in the device such that, advantageously, no physical contact with the skin is needed (or even possible in normal operation), nor a coupling medium (e.g., a gel) between these components and the skin. As such, an interface (e.g., a contact surface, flexible surface, or a platen) may not be present in the sensor apparatus 200, although other wearable or stabilizing structure may be present to secure the sensor apparatus 200 to the user. In some implementations, the sensor apparatus 200 may include a controller system or a controller, which may be configured to perform or cause performance of certain operations. Example configurations and operations will be described in greater detail.

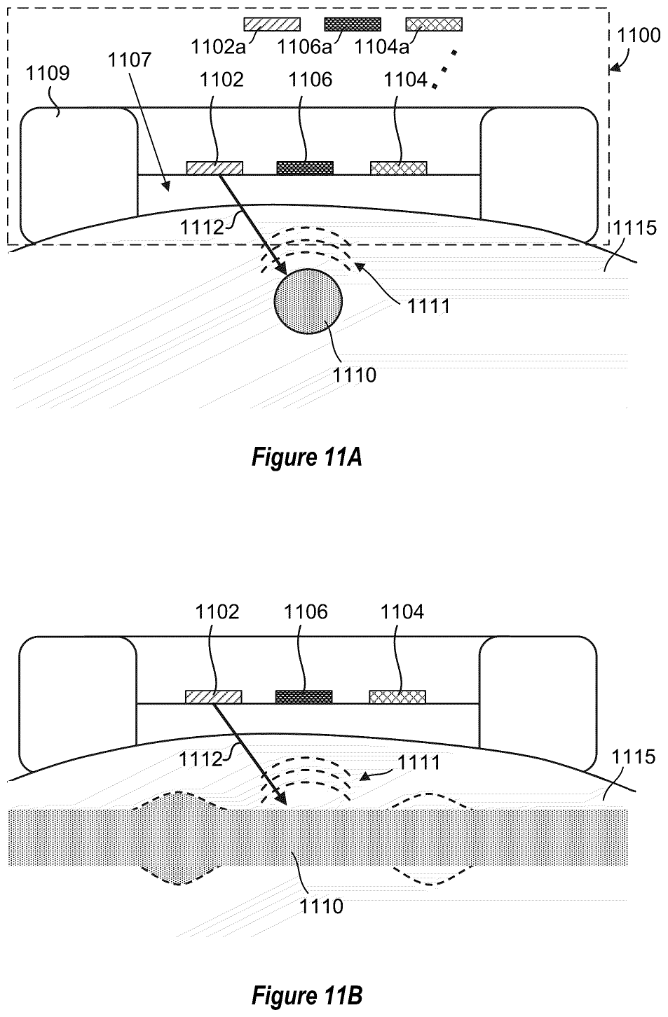

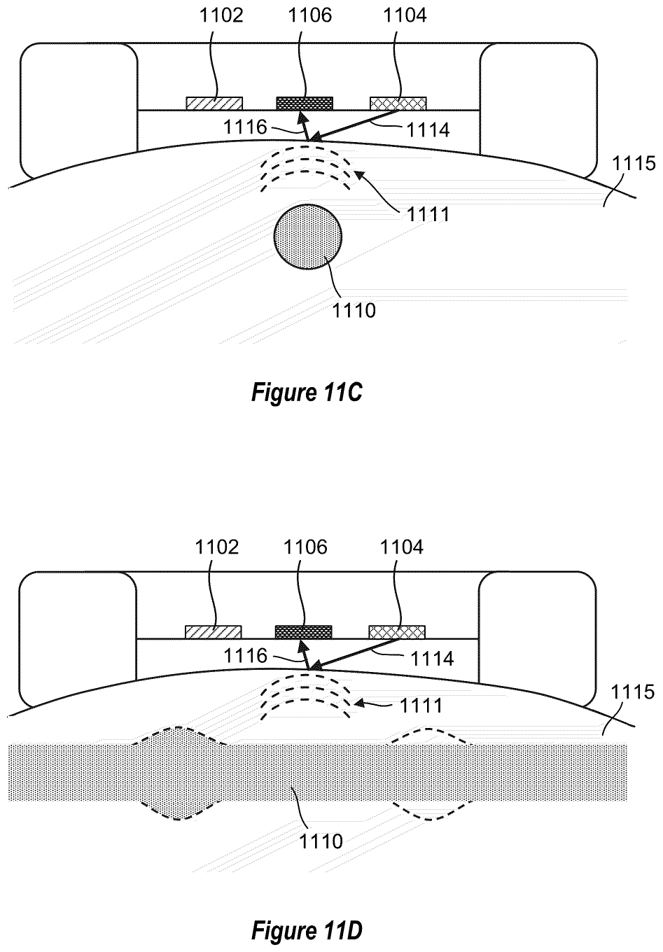

In various embodiments described herein, the first light source system 202, the second light source system 203, the receiver system 204, and at least portions of their components may be implemented with a device (such as a wearable device), which will be described in greater detail with respect to FIGS. 11A-11D.

In some embodiments, the sensor apparatus 200 may be a wearable device configured to be worn by a user, e.g., around the wrist, finger, arm, leg, ankle, waist, car, neck, or another appendage, or another portion of the body. In an example implementation, the sensor apparatus 200 may have the form of a wristwatch and can be worn around the wrist. However, the embodiments described herein are not so limited. In certain cases, the components of the sensor apparatus 200 may not all be worn. For instance, a portion of the sensor apparatus 200 (e.g., the first light source system 202, the second light source system 203, and/or the receiver system 204) may be worn around an appendage, but other components may be in a separate sensor component and/or not be in a wearable chassis.

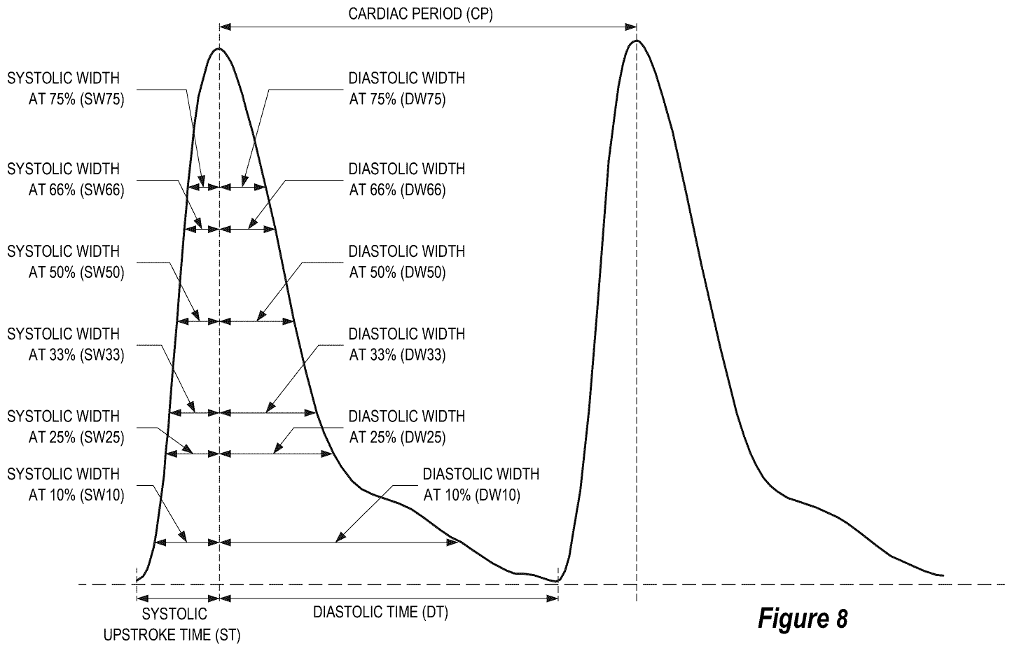

FIG. 8 shows examples of heart rate waveform (HRW) features that may be extracted according to some implementations. The horizontal axis of FIG. 8 represents time and the vertical axis represents signal amplitude. The cardiac period is indicated by the time between adjacent peaks of the HRW. The systolic and diastolic time intervals are indicated below the horizontal axis. During the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

The HRW features that are illustrated in FIG. 8 pertain to the width of the systolic and/or diastolic portions of the HRW curve at various “heights,” which are indicated by a percentage of the maximum amplitude. For example, the SW50 feature is the width of the systolic portion of the HRW curve at a “height” of 50% of the maximum amplitude. In some implementations, the HRW features used for blood pressure estimation may include some or all of the SW10, SW25, SW33, SW50, SW66, SW75, DW10, DW25, DW33, DW50, DW66 and DW75 HRW features. In other implementations, additional HRW features may be used for blood pressure estimation. Such additional HRW features may, in some instances, include the sum and ratio of the SW and DW at one or more “heights,” e.g., (DW75+SW75), DW75/SW75, (DW66+SW66), DW66/SW66, (DW50+SW50), DW50/SW50, (DW33+SW33), DW33/SW33, (DW25+SW25), DW25/SW25 and/or (DW10+SW10), DW10/SW10. Other implementations may use yet other HRW features for blood pressure estimation. Such additional HRW features may, in some instances, include sums, differences, ratios and/or other operations based on more than one “height,” such as (DW75+SW75)/(DW50+SW50), (DW50+SW50/(DW10+SW10), etc.

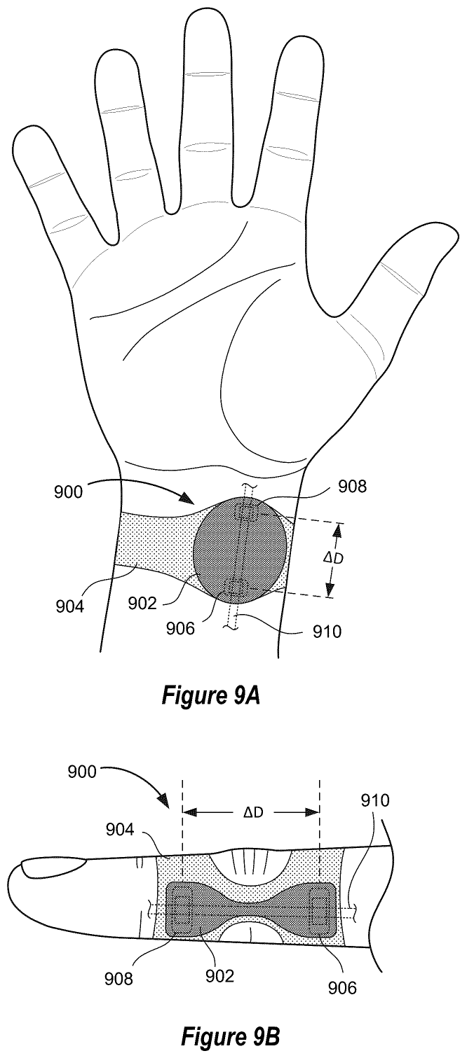

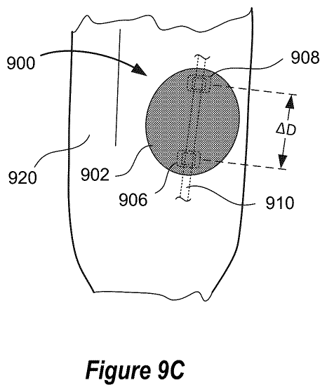

In some implementations, the monitoring device can be positioned around a wrist of a user with a strap or band, similar to a watch or fitness/activity tracker. FIG. 9A shows an example device 900 designed to be worn around a wrist according to some implementations. In some embodiments, the example device 900 may include the sensor apparatus 200 so as to allow components of the sensor apparatus 200 to interact with the user, e.g., via the skin of the user. In the illustrated example, the monitoring device 900 includes a housing 902 integrally formed with, coupled with or otherwise integrated with a wristband 904. The first and the second arterial sensors 906 and 908 may, in some instances, each include an instance of the ultrasonic receiver system and a portion of the light source system that are described above. In this example, the example device 900 is coupled around the wrist such that the first and the second arterial sensors 906 and 908 within the housing 902 are each positioned along a segment of the radial artery 910 (note that the sensors are generally hidden from view from the external or outer surface of the housing facing the subject while the monitoring device is coupled with the subject, but exposed on an inner surface of the housing to enable the sensors to obtain measurements through the subject's skin from the underlying artery). Also as shown, the first and the second arterial sensors 906 and 908 are separated by a fixed distance ΔD. In some other implementations, the example device 900 can similarly be designed or adapted for positioning around a forearm, an upper arm, an ankle, a lower leg, an upper leg, or a finger (all of which are hereinafter referred to as “limbs”) using a strap or band.

FIG. 9B shows an example device 900 designed to be worn on a finger according to some implementations. The first and the second arterial sensors 906 and 908 may, in some instances, each include an instance of the ultrasonic receiver and a portion of the light source system that are described above.

In some other implementations, the devices disclosed herein can be positioned on a region of interest of the user without the use of a strap or band. For example, the first and the second arterial sensors 906 and 908 and other components of the monitoring device can be enclosed in a housing that is secured to the skin of a region of interest of the user using an adhesive or other suitable attachment mechanism (an example of a “patch” monitoring device).

FIG. 9C shows an example device 900 designed to reside on an earbud according to some implementations. According to this example, the monitoring device 900 is coupled to the housing of an earbud 920. The first and second arterial sensors 906 and 908 may, in some instances, each include an instance of the ultrasonic receiver and a portion of the light source system that are described above.

### Example Multi-Source Signal Acquisition and Analyses

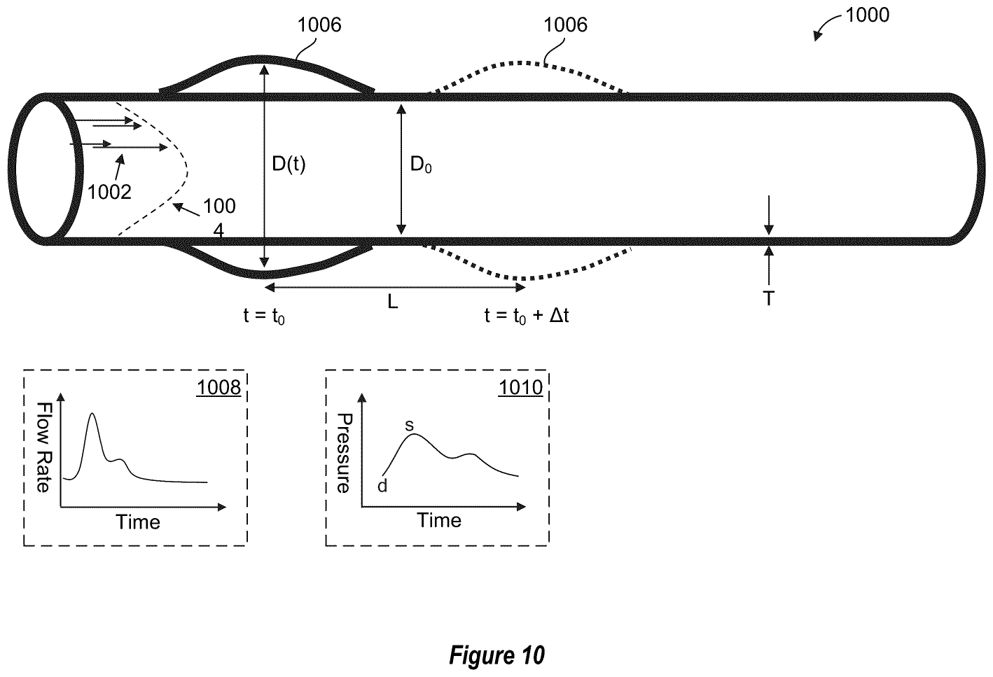

As noted elsewhere, in some applications, a sensor apparatus (e.g., sensor apparatus 200) may be worn at least partially by a user in order to obtain signals relating to a photoacoustic response of a target object such as a blood vessel. FIG. 10 illustrates an example target object 1000 of a user and profiles associated with a pressure wave experienced by the example target object 1000. The example target object 1000 may be a blood vessel in which flow of blood 1002 and its velocity profile 1004 cause distension of the blood vessel and other changes thereto. The blood vessel may have various relevant characteristics and properties that relate to its hyper-elastic, viscoelastic, anisotropic wall. Diameter of the blood vessel at zero strain is denoted as D₀. Diameter of the blood vessel during distension 1006 (e.g., maximum distension) at time t₀ is denoted as D(t). Distension 1006 may be caused at least in part by the flood of blood 1002. Thickness of the wall of the blood vessel is denoted as T. The distension may propagate along the length of the blood vessel. For example, after time Δt has passed from time t₀, the distension 1006 may have traveled a length of L. The blood vessel may further be characterized by a flow rate 1008 over time and a pressure 1010 over time (including systolic(s) and diastolic (d) pressures). Other characteristics of the blood vessel may include, for example, arterial compliance, stiffness, and PWV. As mentioned above, PWV is the velocity of the pressure wave along the arterial wall, and is a relevant factor in determining blood pressure. An example derivation of PWV may be based on measuring photoacoustic signals at two locations separated by a distance D. Two waveforms from two locations may have a time shift t according to PWV. PWV may be estimated as D/t in this case, or ΔD/Δt generally.

FIG. 11A illustrates one view of an example configuration of a multi-light source apparatus 1100 at a first time of operation, according to some embodiments. Apparatus 1100 may be an example of sensor apparatus 200, and may include a first light source 1102, a second light source 1104, and a receiver 1106. The first light source 1102 may be an example of first light source system 202 or a portion thereof, and hence may be configured to emit pulses of optical signals 1112 configured to generate a photoacoustic response (including, e.g., acoustic or ultrasonic waves 1111) from a target object 1110 such as blood vessel. Continuous light may not create a photoacoustic response. The second light source 1104 may be an example of second light source system 203 or a portion thereof, and hence may be configured to emit continuous optical signals that do not generate a photoacoustic response from the target object 1110. The receiver 1106 may be an example of receiver system 204 or a portion thereof, and hence may be configured to detect at least reflected optical signals from the surface of skin 1115, and determine a displacement of the surface of skin 1115, e.g., relative to an undisturbed neutral reference state.

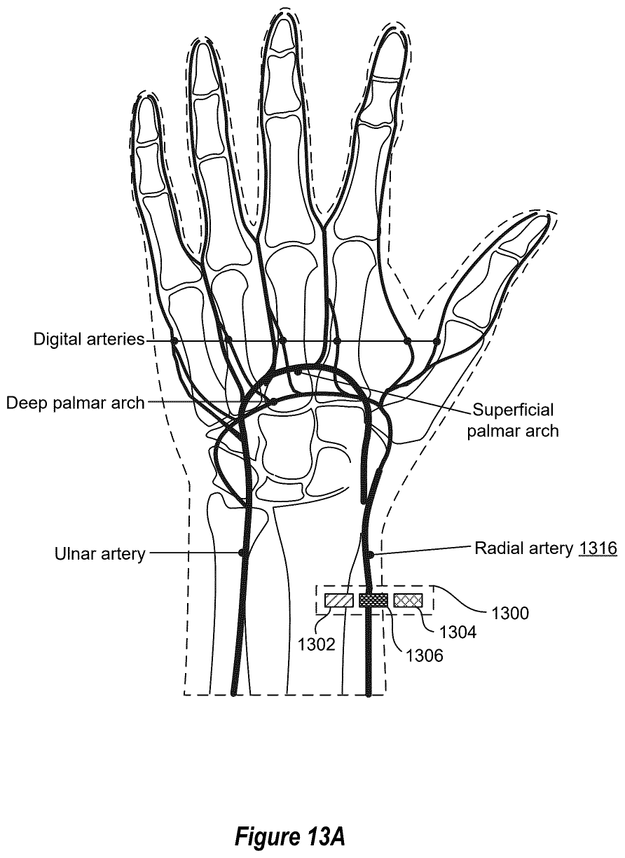

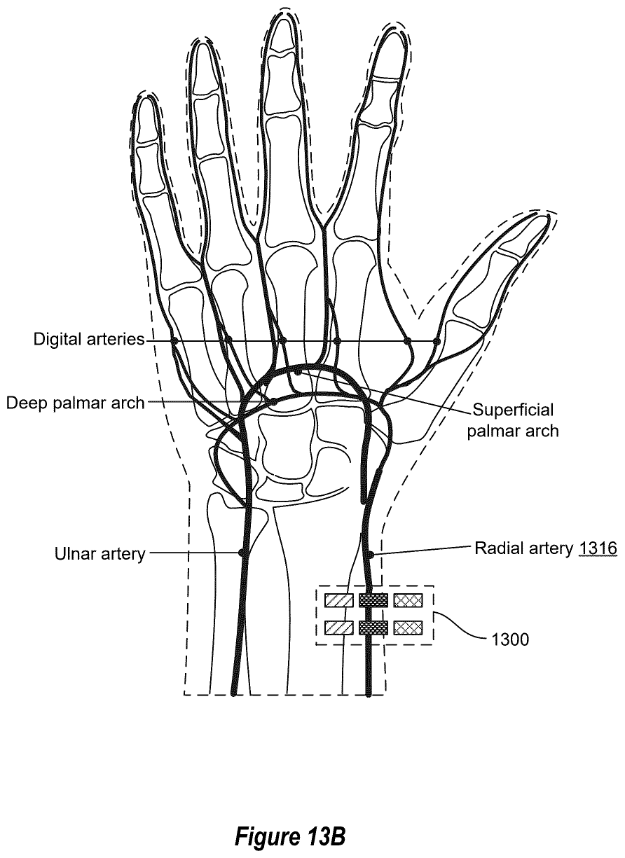

In some configurations, more than one set of light sources and receiver may be used. In some examples, only the first light source 1102, the second light source 1104, and the receiver 1106 may be included in the apparatus 1100. In some examples, at least one additional first light source 1102a, at least one additional second light source 1104b, and at least one additional receiver 1106a may be included, which may be disposed in a location that extends into and out of the illustration. Different placements and quantities of light sources and receivers are possible, of which examples will be described with respect to FIGS. 13A and 13B.

Referring back to FIG. 11A, the apparatus 1100 may include a cavity or an air gap 1107 between the first light source 1102, the second light source 1104, and the receiver 1106, and the surface of skin 1115. The air gap 1107 allows measurement of optical signals in a contactless manner, without a coupling medium (e.g., gel), without applying external pressure on the skin 1115. By not having areas of the skin 1115 being directly touched by a light source or a receiver, any extraneous or environmental pressure or force is removed, at least from relevant portions of the skin 1115, e.g., proximate to where the target object 1110 may generate acoustic waves 1111 and cause motion of the surface of skin 1115. This can result in measurements that are not affected by external pressure because it is possible that such external pressure changes the diameter of the target object 1110. Measurements relating to a disturbed target object may affect measurements relating to its dimensions, and ultimately, characteristics and parameters such as PWV and blood pressure. Hence, the cavity created by the air gap 1107 can ensure there is no external pressure applied to the skin, at least in the relevant sensing region. The skin 1115 may still make contact with or abut a surface of a housing structure 1109 or other parts of a wearable structure. However, such structure may not be contactable where photoacoustic responses (e.g., ultrasonic waves 1111) are generated and portions of the skin 1115 of the user where reflections are measured.

FIG. 11B illustrates another view of the example configuration of a multi-light source apparatus 1100 at the first time of operation, according to some embodiments. More pointedly, while FIG. 11A shows a cross-sectional view of the target object 1110 and skin 1115, FIG. 11B shows a lateral view of the target object 1110 and skin 1115, rotated 90 degrees from the view of FIG. 11A. Similar to in FIG. 11A, pulses of optical signals 1112 may cause a photoacoustic response, and ultrasonic waves 1111 may be generated from the target object 1110. This photoacoustic response may cause vibration or other motion of the surface of the skin 1115.

FIG. 11C illustrates another view of the example configuration of a multi-light source apparatus 1100 at a second time of operation, according to some embodiments. The second time of operation may be subsequent to or prior to the first time of operation illustrated in FIGS. 11A and 11B. The second light source 1104 may be configured to emit a continuous optical signal 1114 toward the surface of the skin 1115. An optical signal 1116 may be reflected from the surface of the skin 1115. During the emission of the continuous optical signal 1114 and the reflection of the optical signal 1116, the first light source 1102 may be emitting pulses of optical signals 1112 configured to trigger a photoacoustic response from the target object 810. The motion of the surface of the skin (its displacement relative to an undisturbed state) caused by the ultrasonic waves 1111 generated as part of the photoacoustic response may be detected by the receiver 1106.

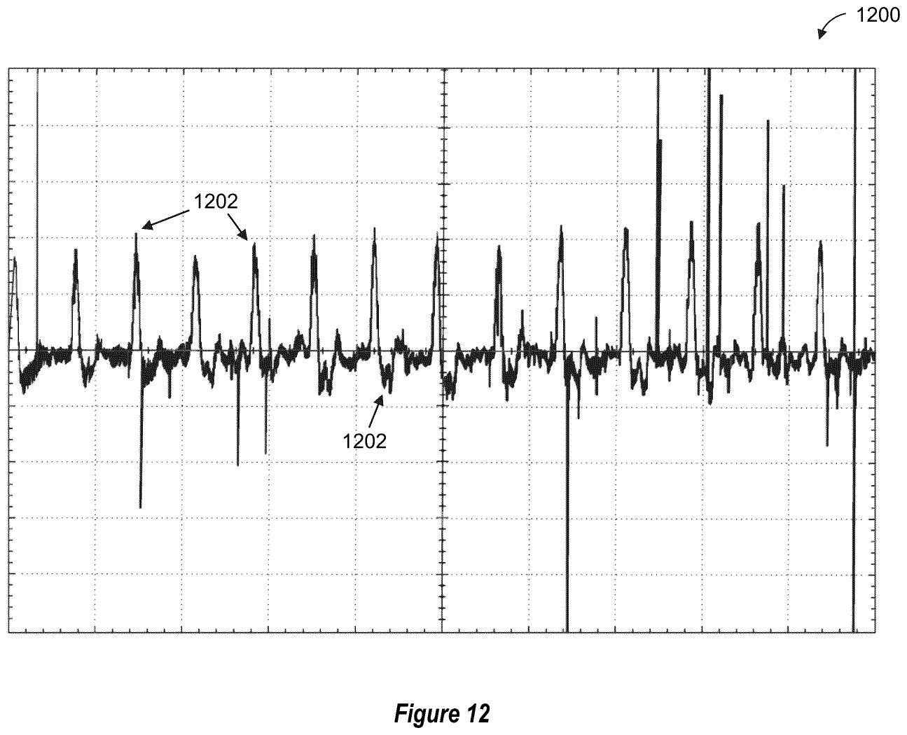

In some approaches, displacement may be determined based on an astigmatic focus differential as described with respect to FIGS. 4A-4C. In some examples, an analog output such as a voltage waveform (such as example waveform 1200 depicted in FIG. 12) may be generated, and information such as amplitude and frequency may be obtained to correlate to the displacement. FIG. 12 depicts an example waveform 1200 representing reflected optical signals measured by a receiver over time. The receiver may be an example of receiver system 204 and include a photosensor such as 312, 410, 520, 620, 622 or 720. In some approaches, amplitudes 1202 may correlate directly with the amount of displacement of an object, such as the surface of skin 1115.

FIG. 11D illustrates another view of the example configuration of a multi-light source apparatus 1100 at the second time of operation, according to some embodiments. While FIG. 11C shows a cross-sectional view of the target object 1110 and skin 1115, FIG. 11D shows a lateral view of the target object 1110 and skin 1115, rotated 90 degrees from the view of FIG. 11C. Similar to in FIG. 11C, a continuous optical signal 1114 may be reflected as optical signal 1116.

In some embodiments, alternatively or additionally to the astigmatic focus differential, a phase difference may be used by the receiver 1106 to decode the displacement of the surface of the skin 1115. The emitted continuous optical signal 1114 may have a first phase, and the reflected optical signal 1116 may have a second phase that is different from the first phase. The difference between the second phase and the first phase may correlate to the motion or position (displacement) of the surface of the skin 1115 caused by a photoacoustic response from the target object 1110, since the second phase of the reflected optical signal 1116 may change depending on the position of the surface of the skin 1115. In addition, or alternatively, the emitted continuous optical signal 1114 may have a first frequency, and the reflected optical signal 1116 may have a second frequency that is different from the first frequency. The difference between the second frequency and the first frequency may result from a Doppler effect and correlate to the motion and velocity of motion of the surface of the skin 1115 caused by a photoacoustic response from the target object 1110. According to implementations, any one or more of the aforementioned approaches and apparatus described with respect to FIGS. 3A-7 may be used to determine the displacement of the surface of the skin 1115.

Amplitude data and other data can be obtained from a continuous analog waveform similar to that shown in FIG. 12 output from the detected reflected optical signal 1116 using a phase difference or a frequency difference. As described herein, motion, and more specifically displacement, of the surface of the skin 1115 can be discerned from amplitude and/or frequency of the collected waveform. The displacement correlates to dimensions (and other characteristics) of the target object 1110 from which the photoacoustic response was obtained. Hence, a focus differential indicative of a distance to the skin, a phase difference associated with the reflection 1116 of the continuous optical signal, a frequency difference associated with the reflection 1116 of the continuous optical signal, or a combination thereof, may be used to determine the displacement.

FIG. 13A shows an example layout of a first light source 1302, a second light source 1304, and a receiver 1306 of a multi-light source apparatus 1300. The multi-light source apparatus 1300 may be an example of apparatus 200 or 1100. The first light source 1302 may be an example of first light source 1102, the second light source 1304 may be an example of first light source 1104, and the receiver 1306 may be an example of receiver 1106. As can be seen, the multi-light source apparatus 1300 may be disposed along or proximate a target object (e.g., blood vessel 1316). In different implementations, the specific arrangement of the first light source 1302, second light source 1306, and receiver 1304 may be varied. That is to say, while the apparatus 1100 and the multi-light source apparatus 1300 shows the receiver 1306 disposed between the first light source 1302 and the second light source 1304, the order and position of these components are not so limited. As but an example, the first light source 1302 and the second light source 1304 may be arranged adjacent to each other, assuming sufficient interference and noise mitigation is in place (e.g., using noise reduction system 210).

Similarly, as shown in FIG. 13B, more than set of the first and second light sources and receiver may be arranged. The example configuration of a multi-light source apparatus 1300 shown here may include two first light sources configured to emit optical signals that obtain a photoacoustic signal from the target object, two second light sources that emit a continuous optical signal, and two receivers that detect reflected optical signals (e.g., reflection of the continuous optical signal), similar to the configuration of FIG. 11A which includes at least one additional first light source 1102a, at least one additional second light source 1104b, and at least one additional receiver 1106a. It will, however, be appreciated that more or fewer of each component may be implemented in any combination, e.g., two first light sources, two second light sources, one receiver. Configurations of the multi-light source apparatus 1300 such as that of FIG. 13B may be useful where a minimum of two sets of a first light source 1302, a second light source 1304, and a receiver 1306 are included and placed at different locations along the length of blood vessel 1316 to be able to estimate PWV. As noted above, ΔD/Δt may be used to estimate PWV.

In some embodiments, an image of objects surrounding the target object may be obtained using the optical signals detected by a receiver system. In some example applications, subdermal structures of tissue surrounding a blood vessel may be generated. Since a light source, such as that configured to emit a continuous optical signal, or a line or an array thereof, can be steered in different directions as mentioned above, a spot or point, a one-dimensional line or a two-dimensional area can be scanned by the light source over time or captured at a time point by the receiver system as photoacoustic responses are created by another light source configured to emit pulses that trigger such responses from a blood vessel. From a one-dimensional line, a two-dimensional cross-section of the tissue can be inferred as the skin vibrates over time. From a two-dimensional area, a three-dimensional representation can be inferred as the skin vibrates over time. From these reconstructed images and representations, structural information can be visually confirmed or determined, e.g., the depth or a diameter of a blood vessel (or other tissue of interest). Another example of photoacoustic imaging is described below.

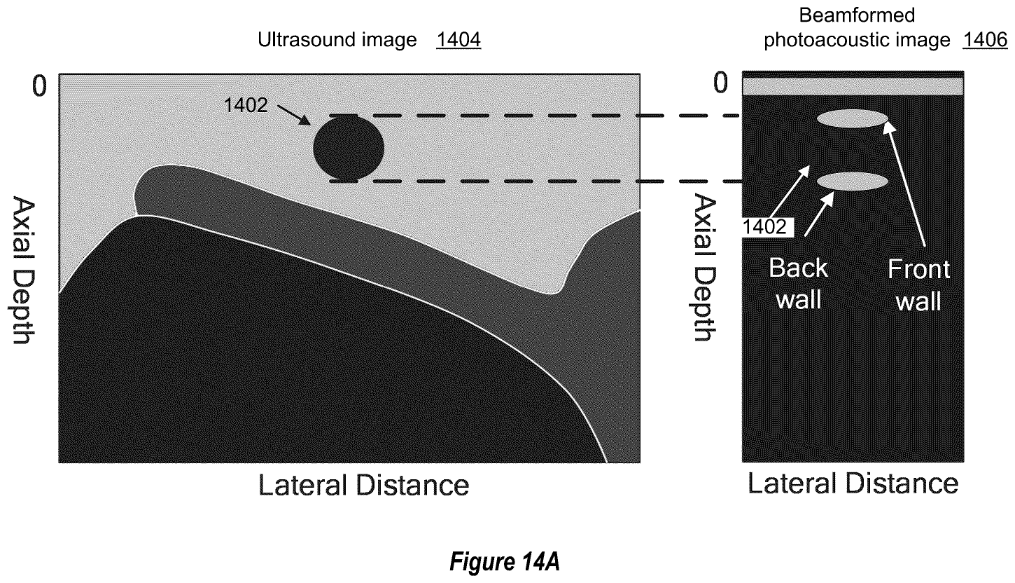

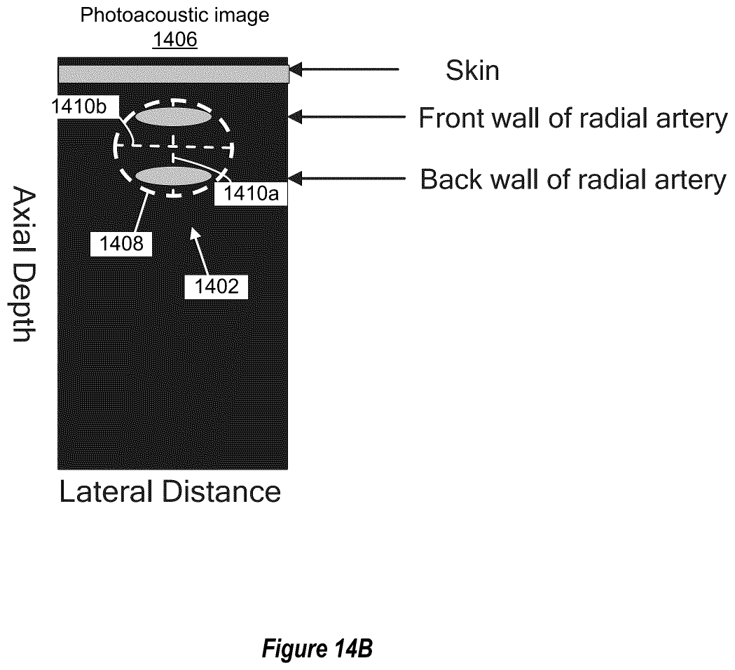

FIG. 14A depicts a beamformed photoacoustic image 1406 based on an ultrasonic image 1404 of a blood vessel 1402, according to some implementations. The ultrasonic image 1404 may depict an image at an arbitrary low-contact pressure. Axial depth (how deep into the tissue) and lateral distance (how wide) of the blood vessel 1402 can be determined from the ultrasonic image 1404 to the photoacoustic image 1406 by correlating edge portions of the blood vessel 1402 in the ultrasonic image 1404 to a back wall and a front wall of the blood vessel 1402 shown in the beamformed photoacoustic image 1406, as indicated by dotted lines.

FIG. 14B depicts the beamformed photoacoustic image 1406 in more detail. The front wall and the back wall of the blood vessel 1402 can be detected in the beamformed photoacoustic image 1406 based on signal strength. In some implementations, an ellipsis or circular boundary 1408 can be fitted onto the beamformed photoacoustic image 1406, and a major axis 1410a and a minor axis 1410b may be determined. Half of the major axis 1410a is referred to as a semi-major axis, and half of the minor axis 1410b is referred to as a semi-minor axis, collectively semi-axes. In some example approaches, image processing, an algorithmic process, or machine learning and training can be used to determine the boundary for the blood vessel 1402, from which axial depth and lateral distance can be used to estimate the major and minor axes. The major and minor axes of the blood vessel 1402 are dimensions that can be used to determine cross-sectional area and/or volume of the blood vessel 1402.

In some embodiments, a machine learning model may be used to predict a physiological characteristic or parameter, e.g., PWV of a blood vessel or a blood pressure of the user. A machine learning model may refer to a computational algorithm that indicates relationships between input variables and output variables. In some embodiments, a machine learning model can be trained. Training a machine learning model may involve, among other things, determining values of weights associated with the machine learning model, where relationships between the input variables and the output variables are based at least in part on the determined weight values. In one implementation, a machine learning model may be trained in a supervised manner using a training set that includes labeled training data. In a more particular example, the labeled training data may include inputs and manually annotated outputs that the machine learning model is to approximate using determined weight values. In another implementation, a machine learning model may be trained in an unsupervised manner in which weight values are determined without manually labeled training data.

An example training process for the machine learning model may involve providing training data that includes, for example, a range of wavelengths or selected wavelengths used for continuous optical signals, known amplitude (and/or frequency) data of reflections of optical signals at certain wavelengths (e.g., those used for continuous optical signals emitted to skin), images of subdermal structures reconstructed by steering light source(s), and/or known arterial spatial measurements (e.g., diameter), as well as the “ground truth” or the known output characteristics or parameters, e.g., PWV, blood pressure, or cardiac features (e.g., peaks or HRW features) that are known. In some approaches, a portion (e.g., 20%) of the training data may be used as part of a validation set for the machine learning model. With this training data and validation set, one or more loss functions may be implemented. A loss function is an optimization function in which an error is iteratively minimized through, e.g., gradient descent. A productive learning rate that dictates the “step” the gradient descent takes when finding the lowest error may be set during training as well.

As a result, a trained machine learning model can be generated. In some implementations, such a trained machine learning model can be used to further enhance the accuracy and reliability of the estimated physiological characteristics or parameter. For example, an estimation derived from measurements from the disclosed apparatus and various derivations based on the measurements can be provided to the machine learning model (stored at a sensor or a host device and/or accessible by a control system thereof) to compare with a physiological characteristic of a blood vessel (e.g., PWV, heart rate) or a physiological parameter of a user (e.g., blood pressure) estimated by the machine learning model. If there is a discrepancy between the sensor-based estimation and the model-generated prediction which is greater than a threshold, the obtained estimation may be further evaluated or discarded. If discarded, the model-generated prediction may be used, or additional measurements may be taken by the sensors. In cases where there are more than two sensors on the user and a discrepancy arises between the sensor estimations and the model predictions, fewer sensors may be used as a fallback rather than all sensors. On the other hand, if the discrepancy is lower than a threshold, the sensor-based estimation may be selected or kept for further processing, sending to a host device, reporting, displaying to the user, etc.

### Example Methods

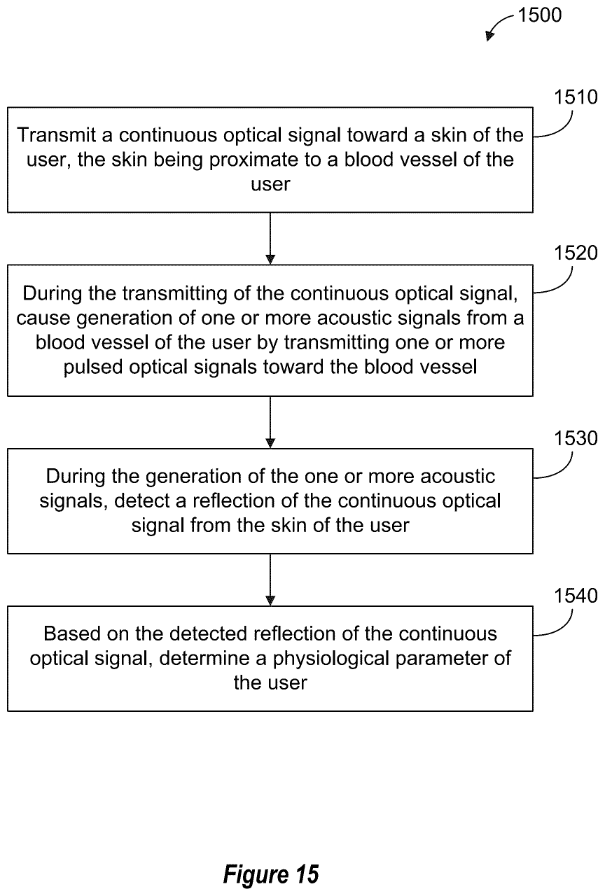

FIG. 15 is a flow diagram of a method 1500 of determining a physiological parameter of a user, according to some disclosed embodiments. Structure for performing the functionality illustrated in one or more of the blocks shown in FIG. 15 may be performed by hardware and/or software components of a computerized apparatus or system (which may be implemented as a wearable device in some embodiments). Components of such apparatus or system may include, for example, a first light source, a second light source, a receiver, a control system (including one or more processors), a memory, and/or a computer-readable apparatus including a storage medium storing computer-readable and/or computer-executable instructions that are configured to, when executed by the control system, cause the control system, the one or more processors, or the apparatus to perform operations represented by blocks below. Example components of the apparatus are illustrated in, e.g., FIGS. 2 and 11A-11D, which are described in more detail above.

The blocks of FIG. 15 may, for example, be performed by the apparatus 200, 1100, 1300 or by a similar apparatus, or a component thereof (e.g., a control system). As with other methods disclosed herein, the method outlined in FIG. 15 may include more or fewer blocks than indicated. Moreover, the blocks of methods disclosed herein are not necessarily performed in the order indicated. In some instances, one or more of the blocks shown in FIG. 15 may be performed concurrently.

At block 1510, the method 1500 may include transmitting a continuous optical signal toward a skin of the user, the skin being proximate to a blood vessel of the user. In some examples, the continuous optical signal may be emitted by a laser diode or an LED.

Means for performing functionality at block 1510 may include the second light source system 203 and/or other components of the apparatus as shown in FIG. 2.

At block 1520, the method 1500 may include, during the transmitting of the continuous optical signal, causing generation of one or more acoustic signals from a blood vessel of the user by transmitting one or more pulsed optical signals toward the blood vessel. In some examples, the one or more pulsed optical signals may be emitted by a laser diode or an LED configured to trigger a photoacoustic response from the blood vessel.

Means for performing functionality at block 1520 may include the first light source system 202 and/or other components of the apparatus as shown in FIG. 2.

At block 1530, the method 1500 may include, during the generation of the one or more acoustic signals, detecting a reflection of the continuous optical signal from the skin of the user. In some embodiments, the one or more pulsed optical signals may include one or more optical signals emitted by a first light source; the continuous optical signal may include a continuous optical signal emitted by a second light source; and the first light source and the second light source may be disposed at different locations of a device. In some implementations, the device may include a wearable device, and may further include a receiver configured to detect the reflection of the continuous optical signal.

In some embodiments, the continuous optical signal emitted by the second light source may include an optical signal having a wavelength of up to 1550 nm generated by a second laser over a first duration up to 10 microseconds; and the one or more pulsed optical signals emitted by the first light source may include one or more optical signals having a wavelength of 450-850 nm each generated by a first laser configured to obtain a photoacoustic response from the blood vessel over a second duration up to 500 nanoseconds during the first duration.

Means for performing functionality at block 1530 may include, the receiver system 204 (e.g., photosensor 312, 410, 520, 620, 622 or 720) and/or other components of the apparatus as shown in FIG. 2.

At block 1540, the method 1500 may include, based on the detected reflection of the continuous optical signal, determining a physiological parameter of the user.

In some embodiments, the method 1500 may further include determining a physical characteristic of the blood vessel based at least on the reflection of the continuous optical signal. In some implementations, the determining of the physiological parameter of the user may be based on the physical characteristic of the blood vessel.

In some embodiments, a control system may be configured to determine a physical characteristic of the target object based at least on the reflection of the first optical signal; and determine the physiological parameter of the user based on the physical characteristic of the target object.

In some implementations, the physical characteristic of the blood vessel may include a distension experienced by the blood vessel during the generation of the one or more acoustic signals, or a pulse wave velocity (PWV) of the blood vessel. In some implementations, the physiological parameter of the user may include a blood pressure of the user.

In some implementations, the determining of the physical characteristic of the target object or blood vessel may include determining a displacement of the skin of the user, the physical characteristic of the blood vessel determined based on the displacement of the skin. In some variants, the determining of the displacement of the skin of the user may include using a focus differential indicative of a distance to the skin, a frequency difference associated with the reflection of the continuous optical signal, a phase difference associated with the reflection of the continuous optical signal, or a combination thereof.

In some embodiments, the method 1500 may further include obtaining an image representation of at least the reflection of the continuous optical signal. In some examples, the image representation may be a two-dimensional cross-section of tissue, a three-dimensional representation of tissue, or a photoacoustic image (e.g., 1406). In some implementations, the determining of the physical characteristic of the blood vessel may be based on the image representation.

Means for performing functionality at block 1540 may include the receiver system 204 (e.g., photosensor 312, 410, 520, 620, 622 or 720), the control system 206, and/or other components of the apparatus as shown in FIG. 2.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also may be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that may be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those having ordinary skill in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “exemplary” is used exclusively herein, if at all, to mean “serving as an example, instance, or illustration.” Any implementation described herein as “exemplary” is not necessarily to be construed as preferred or advantageous over other implementations.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

It will be understood that unless features in any of the particular described implementations are expressly identified as incompatible with one another or the surrounding context implies that they are mutually exclusive and not readily combinable in a complementary and/or supportive sense, the totality of this disclosure contemplates and envisions that specific features of those complementary implementations may be selectively combined to provide one or more comprehensive, but slightly different, technical solutions. It will therefore be further appreciated that the above description has been given by way of example only and that modifications in detail may be made within the scope of this disclosure.

Various modifications to the implementations described in this disclosure may be readily apparent to those skilled in the art, and the generic principles defined herein may be applied to other implementations without departing from the spirit or scope of this disclosure. Thus, the following claims are not intended to be limited to the implementations shown herein, but are to be accorded the widest scope consistent with this disclosure, the principles and the novel features disclosed herein.

Additionally, certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. Moreover, various ones of the described and illustrated operations can itself include and collectively refer to a number of sub-operations. For example, each of the operations described above can itself involve the execution of a process or algorithm. Furthermore, various ones of the described and illustrated operations can be combined or performed in parallel in some implementations. Similarly, the separation of various system components in the implementations described above should not be understood as requiring such separation in all implementations. As such, other implementations are within the scope of the following claims. In some cases, the actions recited in the claims can be performed in a different order and still achieve desirable results.

Implementation examples are described in the following numbered clauses:

Clause 1: A method of determining a physiological parameter of a user, the method including: transmitting a continuous optical signal toward a skin of the user, the skin being proximate to a blood vessel of the user; during the transmitting of the continuous optical signal, causing generation of one or more acoustic signals from a blood vessel of the user by transmitting one or more pulsed optical signals toward the blood vessel; during the generation of the one or more acoustic signals, detecting a reflection of the continuous optical signal from the skin of the user; and based on the detected reflection of the continuous optical signal, determining a physiological parameter of the user.

Clause 2: The method of clause 1, further including determining a physical characteristic of the blood vessel based at least on the reflection of the continuous optical signal; wherein the determining of the physiological parameter of the user is based on the physical characteristic of the blood vessel.

Clause 3: The method of clause 2, wherein: the physical characteristic of the blood vessel includes a distension experienced by the blood vessel during the generation of the one or more acoustic signals, or a pulse wave velocity (PWV) of the blood vessel; and the physiological parameter of the user includes a blood pressure of the user.

Clause 4: The method of clause 2, further including obtaining an image representation of at least the reflection of the continuous optical signal; wherein the determining of the physical characteristic of the blood vessel is based on the image representation.

Clause 5: The method of clause 2, wherein the determining of the physical characteristic of the blood vessel includes determining a displacement of the skin of the user, the physical characteristic of the blood vessel determined based on the displacement of the skin.

Clause 6: The method of clause 5, wherein the determining of the displacement of the skin of the user includes using a focus differential indicative of a distance to the skin, a frequency difference associated with the reflection of the continuous optical signal, a phase difference associated with the reflection of the continuous optical signal, or a combination thereof.

Clause 7: The method of clause 1, wherein: the one or more pulsed optical signals includes one or more optical signals emitted by a first light source; the continuous optical signal includes a continuous optical signal emitted by a second light source; and the first light source and the second light source are disposed at different locations of a device.

Clause 8: The method of clause 7, wherein the device includes a wearable device, and further includes a receiver configured to detect the reflection of the continuous optical signal.

Clause 9: The method of clause 7, wherein: the continuous optical signal emitted by the second light source includes an optical signal having a wavelength of up to 1550 nm generated by a second laser over a first duration up to 10 microseconds; and the one or more pulsed optical signals emitted by the first light source include one or more optical signals having a wavelength of 450-850 nm each generated by a first laser configured to obtain a photoacoustic response from the blood vessel over a second duration up to 500 nanoseconds during the first duration.

Clause 10: A user device including: a first light source system configured to transmit one or more first optical signals toward the target object, the one or more second optical signals configured to generate one or more acoustic signals from the a target object; a second light source system configured to transmit a second optical signal toward a skin of the user, the skin being proximate to the target object of the user; a receiver configured to detect a reflection of the second optical signal from the skin of the user during the generation of the one or more acoustic signals, the reflection of the second optical signal indicative of a physiological parameter of the user; and a wearable structure securable to the user and including the first light source system, the second light source system, and the receiver.

Clause 11: The user device of clause 10, further including a control system, the control system configured to: determine a physical characteristic of the target object based at least on the reflection of the second optical signal; and determine the physiological parameter of the user based on the physical characteristic of the target object.

Clause 12: The user device of clause 11, wherein: the physical characteristic of the target object including a distension experienced by the target object during the generation of the one or more acoustic signals, or a pulse wave velocity (PWV) of the target object; and the physiological parameter of the user includes a blood pressure of the user.

Clause 13: The user device of clause 11, wherein the determination of the physical characteristic of the target object includes determination of a displacement of the skin, the physical characteristic of the target object determined based on the displacement of the skin.

Clause 14: The user device of clause 13, wherein the determination of the displacement of the skin of the user is based on a focus differential indicative of a distance to the skin, a change in frequency associated with the reflection of the second optical signal, a phase difference associated with the reflection of the second optical signal, or a combination thereof.

Clause 15: The user device of clause 10, wherein: the target object includes a blood vessel of the user; the one or more first optical signals include pulsed laser signals; the second optical signal includes a continuous laser signal; and the receiver includes a photodetector configured to determine a frequency or a phase of the reflection of the continuous laser signal while the pulsed laser signals are transmitted toward the target object of the user.

Clause 16: The user device of clause 15, further including a control system, the control system configured to determine the physiological parameter of the user based on a displacement of the skin during the generation of the one or more acoustic signals, the displacement of the skin determined based on a focus differential indicative of a distance to the skin, a difference in the frequency associated with the reflection of the continuous laser signal, a phase difference associated with the reflection of the continuous laser signal, or a combination thereof.

Clause 17: A non-transitory computer-readable apparatus including a storage medium, the storage medium including a plurality of instructions configured to, when executed by a control system, cause an apparatus to: transmit a continuous optical signal toward a skin of the user, the skin being proximate to a blood vessel of the user; during the transmitting of the continuous optical signal, cause generation of one or more acoustic signals from a blood vessel of the user by transmitting one or more pulsed optical signals toward the blood vessel; during the generation of the one or more acoustic signals, detect a reflection of the continuous optical signal from the skin of the user; and based on the detected reflection of the continuous optical signal, determine a physiological parameter of the user.

Clause 18: The non-transitory computer-readable apparatus of clause 17, wherein the plurality of instructions are further configured to, when executed by the control system, cause the apparatus to: determine a physical characteristic of the blood vessel based at least on the reflection of the continuous optical signal, wherein the physical characteristic of the blood vessel includes a distension experienced by the blood vessel during the generation of the one or more acoustic signals, or a pulse wave velocity (PWV) of the blood vessel; and determine the physiological parameter of the user based on the physical characteristic of the blood vessel, wherein the physiological parameter of the user includes a blood pressure of the user.

Clause 19: The non-transitory computer-readable apparatus of clause 17, wherein the determination of the physical characteristic of the blood vessel includes determination of a displacement of the skin of the user using a focus differential indicative of a distance to the skin, a frequency difference associated with the reflection of the continuous optical signal, a phase difference associated with the reflection of the continuous optical signal, or a combination thereof.

Clause 20: The non-transitory computer-readable apparatus of clause 17, wherein: the continuous optical signal includes a continuous optical signal emitted by a second light source, wherein the continuous optical signal emitted by the second light source includes an optical signal having a wavelength of up to 1550 nm generated by a second laser over a first duration up to 10 microseconds; and the one or more pulsed optical signals include one or more optical signals emitted by a first light source, wherein the one or more pulsed optical signals emitted by the first light source include one or more optical signals having a wavelength of 500-850 nm each generated by a first laser configured to obtain a photoacoustic response from the blood vessel over a second duration up to 500 nanoseconds during the first duration; and the first light source and the second light source are disposed at different locations of a device.

What is claimed:

1. A method of determining a physiological parameter of a user, the method comprising:
transmitting a continuous optical signal toward a skin of the user, the skin being proximate to a blood vessel of the user;
during the transmitting of the continuous optical signal, causing generation of one or more acoustic signals from a blood vessel of the user by transmitting one or more pulsed optical signals toward the blood vessel;
during the generation of the one or more acoustic signals, detecting a reflection of the continuous optical signal from the skin of the user; and
based on the detected reflection of the continuous optical signal, determining a physiological parameter of the user.

2. The method of claim 1, further comprising determining a physical characteristic of the blood vessel based at least on the reflection of the continuous optical signal;
wherein the determining of the physiological parameter of the user is based on the physical characteristic of the blood vessel.

3. The method of claim 2, wherein:
the physical characteristic of the blood vessel comprises a distension experienced by the blood vessel during the generation of the one or more acoustic signals, or a pulse wave velocity (PWV) of the blood vessel; and
the physiological parameter of the user comprises a blood pressure of the user.

4. The method of claim 2, further comprising obtaining an image representation of at least the reflection of the continuous optical signal;
wherein the determining of the physical characteristic of the blood vessel is based on the image representation.

5. The method of claim 2, wherein the determining of the physical characteristic of the blood vessel comprises determining a displacement of the skin of the user, the physical characteristic of the blood vessel determined based on the displacement of the skin.

6. The method of claim 5, wherein the determining of the displacement of the skin of the user comprises using a focus differential indicative of a distance to the skin, a frequency difference associated with the reflection of the continuous optical signal, a phase difference associated with the reflection of the continuous optical signal, or a combination thereof.

7. The method of claim 1, wherein:
the one or more pulsed optical signals comprise one or more optical signals emitted by a first light source;
the continuous optical signal comprises a continuous optical signal emitted by a second light source; and
the first light source and the second light source are disposed at different locations of a device.

8. The method of claim 7, wherein the device comprises a wearable device, and further comprises a receiver configured to detect the reflection of the continuous optical signal.

9. The method of claim 7, wherein:
the continuous optical signal emitted by the second light source comprises an optical signal having a wavelength of up to 1550 nm generated by a second laser over a first duration up to 10 microseconds; and
the one or more pulsed optical signals emitted by the first light source comprise one or more optical signals having a wavelength of 450-850 nm each generated by a first laser configured to obtain a photoacoustic response from the blood vessel over a second duration up to 500 nanoseconds during the first duration.

10. A user device comprising:
a first light source system configured to transmit one or more first optical signals toward the target object, the one or more second optical signals configured to generate one or more acoustic signals from the a target object;
a second light source system configured to transmit a second optical signal toward a skin of the user, the skin being proximate to the target object of the user;
a receiver configured to detect a reflection of the second optical signal from the skin of the user during the generation of the one or more acoustic signals, the reflection of the second optical signal indicative of a physiological parameter of the user; and
a wearable structure securable to the user and comprising the first light source system, the second light source system, and the receiver.

11. The user device of claim 10, further comprising a control system, the control system configured to:
determine a physical characteristic of the target object based at least on the reflection of the second optical signal; and
determine the physiological parameter of the user based on the physical characteristic of the target object.

12. The user device of claim 11, wherein:
the physical characteristic of the target object comprises a distension experienced by the target object during the generation of the one or more acoustic signals, or a pulse wave velocity (PWV) of the target object; and
the physiological parameter of the user comprises a blood pressure of the user.

13. The user device of claim 11, wherein the determination of the physical characteristic of the target object comprises determination of a displacement of the skin, the physical characteristic of the target object determined based on the displacement of the skin.

14. The user device of claim 13, wherein the determination of the displacement of the skin of the user is based on a focus differential indicative of a distance to the skin, a change in frequency associated with the reflection of the second optical signal, a phase difference associated with the reflection of the second optical signal, or a combination thereof.

15. The user device of claim 10, wherein:
the target object comprises a blood vessel of the user;
the one or more first optical signals comprise pulsed laser signals;
the second optical signal comprises a continuous laser signal; and
the receiver comprises a photodetector configured to determine a frequency or a phase of the reflection of the continuous laser signal while the pulsed laser signals are transmitted toward the target object of the user.

16. The user device of claim 15, further comprising a control system, the control system configured to determine the physiological parameter of the user based on a displacement of the skin during the generation of the one or more acoustic signals, the displacement of the skin determined based on a focus differential indicative of a distance to the skin, a difference in the frequency associated with the reflection of the continuous laser signal, a phase difference associated with the reflection of the continuous laser signal, or a combination thereof.

17. A non-transitory computer-readable apparatus comprising a storage medium, the storage medium comprising a plurality of instructions configured to, when executed by a control system, cause an apparatus to:
transmit a continuous optical signal toward a skin of the user, the skin being proximate to a blood vessel of the user;
during the transmitting of the continuous optical signal, cause generation of one or more acoustic signals from a blood vessel of the user by transmitting one or more pulsed optical signals toward the blood vessel;
during the generation of the one or more acoustic signals, detect a reflection of the continuous optical signal from the skin of the user; and
based on the detected reflection of the continuous optical signal, determine a physiological parameter of the user.

18. The non-transitory computer-readable apparatus of claim 17, wherein the plurality of instructions are further configured to, when executed by the control system, cause the apparatus to:
determine a physical characteristic of the blood vessel based at least on the reflection of the continuous optical signal, wherein the physical characteristic of the blood vessel comprises a distension experienced by the blood vessel during the generation of the one or more acoustic signals, or a pulse wave velocity (PWV) of the blood vessel; and
determine the physiological parameter of the user based on the physical characteristic of the blood vessel, wherein the physiological parameter of the user comprises a blood pressure of the user.

19. The non-transitory computer-readable apparatus of claim 17, wherein the determination of the physical characteristic of the blood vessel comprises determination of a displacement of the skin of the user using a focus differential indicative of a distance to the skin, a frequency difference associated with the reflection of the continuous optical signal, a phase difference associated with the reflection of the continuous optical signal, or a combination thereof.

20. The non-transitory computer-readable apparatus of claim 17, wherein:
the continuous optical signal comprises a continuous optical signal emitted by a second light source, wherein the continuous optical signal emitted by the second light source comprises an optical signal having a wavelength of up to 1550 nm generated by a second laser over a first duration up to 10 microseconds; and
the one or more pulsed optical signals comprise one or more optical signals emitted by a first light source, wherein the one or more pulsed optical signals emitted by the first light source comprise one or more optical signals having a wavelength of 450-850 nm each generated by a first laser configured to obtain a photoacoustic response from the blood vessel over a second duration up to 500 nanoseconds during the first duration; and
the first light source and the second light source are disposed at different locations of a device.
